SEQUENCE LISTING (3 sequences)

SEQ ID NO: 1 - protein, 1296 aa, Clostridium botulinum
  source 1..1296: note = amino acid sequence of the 150 kDa BoNT/A molecule
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 437 aa, Clostridium botulinum
  source 1..437: note = amino acid sequence of the light chain (LC) of BoNT/A molecule
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 3 - protein, 848 aa, Clostridium botulinum
  source 1..848: note = amino acid sequence of the heavy chain (HC) of BoNT/A molecule
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

(19) United States
(12) Patent Application Publication
(10) Pub. No.: US 2025/0235515 A1
(43) Pub. Date: Jul. 24, 2025

(54) CLOSTRIDIUM BOTULINUM NEUROTOXIN SEROTYPE A COMPOSITIONS AND IMPROVED DRYING PROCESSES

(71) Applicant: ABBVIE INC., North Chicago, IL (US)

(72) Inventors: Evgenyi SHALAEV, Dana Point, CA (US); Maurice ABIAD, Long Beach, CA (US)

(73) Assignee: ABBVIE INC., North Chicago, IL (US)

(21) Appl. No.: 19/029,923

(22) Filed: Jan. 17, 2025

Related U.S. Application Data
(60) Provisional application No. 63/623,130, filed on Jan. 19, 2024.

(51) Int. Cl. A61K 38/48; A61K 38/38; C07K 14/765

(52) U.S. Cl. CPC A61K 38/4893; A61K 38/385; C07K 14/765; C12Y 304/24069

(57) ABSTRACT

Provided herein are solid compositions comprising Clostridium botulinum neurotoxin serotype A (BoNT/A) and human serum albumin (HSA), wherein the solid compositions comprise a low amount of HSA aggregates. Further provided herein are methods of producing solid compositions comprising BoNT/A and HSA by drying a solution comprising BoNT/A in a manner such that the solution does not undergo freezing during drying.

### CROSS-REFERENCE TO RELATED APPLICATIONS

This application claims the benefit of U.S. Provisional Patent Application No. 63/623,130, filed Jan. 19, 2024, the disclosure of which is incorporated by reference herein in its entirety.

### REFERENCE TO SEQUENCE LISTING SUBMITTED ELECTRONICALLY

This application contains an electronic Sequence Listing which has been submitted in XML file format via Patent Center, the entire content of which is incorporated by reference herein in its entirety. The Sequence Listing XML file submitted with this application is entitled “13371-306-999_SEQLISTING.xml”, was created on Jan. 3, 2024, and is 6,736 bytes in size.

### 1. FIELD

Provided herein are solid compositions comprising Clostridium botulinum neurotoxin serotype A (BoNT/A) and human serum albumin (HSA), wherein the solid compositions comprise a low amount of HSA aggregates. Further provided herein are methods of producing solid compositions comprising BoNT/A and HSA by drying a solution comprising BoNT/A in a manner such that the solution does not undergo freezing during drying.

### 2. BACKGROUND

Clostridium botulinum neurotoxin serotype A (BoNT/A) is a highly potent toxin that causes muscle relaxation by inhibition of synaptic vesicle docking and fusion, thereby blocking acetylcholine release at neuromuscular junctions. BoNT/A is produced by Clostridium botulinum Type A strains, which synthesize a complex of a 150 kDa neurotoxin, along with a group of non-toxic neurotoxin-associated proteins (NAPs). BOTOX®, or onabotulinumtoxinA, is a BoNT/A product approved by the United States Food and Drug Administration (FDA) in 1989 for a variety of therapeutic and cosmetic indications.

Manufacturing protein-based pharmaceutical compositions for biopharmaceutical applications involves multiple steps. Certain parameters of some manufacturing steps may affect the properties or functionalities of the proteins.

To date, there remains a need for developing botulinum toxins pharmaceutical compositions with improved properties.

Citation of a reference herein shall not be construed as an admission that such is prior art to the present disclosure.

### 3. SUMMARY OF THE INVENTION

The present disclosure provides novel solid compositions comprising Clostridium botulinum neurotoxin serotype A (BoNT/A) with advantageous properties, as well as novel processes for producing such compositions.

More specifically, the present disclosure provides solid compositions comprising Clostridium botulinum neurotoxin serotype A (BoNT/A) and human serum albumin (HSA), wherein the solid compositions comprise a low amount of HSA aggregates. The present disclosure also provides methods of producing solid compositions comprising BoNT/A and HSA, wherein the methods comprise a step of drying a solution comprising BoNT/A and HSA to produce said solid composition, wherein the solution does not undergo freezing during the drying step. The present disclosure further provides solid compositions that are produced by such a method.

HSA has been used as an excipient in protein therapeutic formulations to improve stability and resilience to external stresses, by, for example, preventing aggregation, oxidation, and surface adsorption. HSA serves as a particularly important stabilizer for BOTOX®'s low fill volume-high potency formulation, given its ability to potentially reduce the unfolding and aggregation of BoNT/A partially due to its surface covering property. Without wishing to be bound by any one theory, the present disclosure describes that freezing during drying unexpectedly results in a significant increase in the amount of HSA aggregation in a solid BoNT/A composition, as compared to drying without freezing, that certain measures may be employed to avoid freezing during vacuum drying of BoNT/A compositions, and that solid BoNT/A compositions dried without undergoing freezing have beneficial properties such as consistent visual product quality and a much slower potency decline during storage, e.g., at room temperature or higher. Accordingly, the present invention provides novel solid compositions comprising BoNT/A and a low amount of HSA aggregates as well as manufacturing methods with an improved drying process for obtaining such solid compositions.

In one aspect, provided herein is a solid composition comprising BoNT/A and HSA, wherein the percentage of HSA aggregates, HSA polymer, HSA oligomer, or HSA dimer in soluble HSA contained in the solid composition is reduced. Specifically, provided herein is a solid composition comprising a 900 kDa BoNT/A complex, HSA, and sodium chloride (NaCl), wherein the HSA comprises HSA monomer and HSA aggregates, wherein at least a portion of the HSA is in the form of soluble HSA when the solid composition is reconstituted in an aqueous medium, and wherein less than 34% of soluble HSA contained in the solid composition are HSA aggregates.

Also provided herein is a method of producing a solid composition comprising a 900 kDa BoNT/A complex, HSA, and NaCl, said method comprising a step of drying a solution comprising BoNT/A, HSA, and NaCl to produce said solid composition, wherein the solution does not undergo freezing during the drying step.

In another aspect, provided herein is a solid composition produced by a method described herein.

In another aspect, provided herein is a method of treating a patient in need thereof, comprising administering a solid composition described herein.

### 4. BRIEF DESCRIPTION OF FIGURES

FIG. 1. A copy of a part of the Certificate of Analysis for an HSA lot, CSL Bering.

FIG. 2A. Representative vacuum drying cycle data. Product (lot #4) temperature was measured by Ellab temperature probes.

FIG. 2B. Representative freeze-drying cycles data. Product (lot #9) temperature was measured by Ellab temperature probes.

FIGS. 3A-3C. Representative SEC traces for HSA collected using different detection methods: fluorescence (excitation 280 nm/emission 350 nm) (FIG. 3A), UV 280 nm (FIG. 3B), and UV 220 nm (FIG. 3C).

FIG. 4A. Representative SEC traces for reconstituted products produced by vacuum drying (process-1), freeze-drying (process-2), and freeze-drying with pre- and post-filtration hold (process-3) (UV 220 nm detector). Inset shows graph of percent aggregation in the form of HSA polymer (left bars) and HSA dimer (right bars) in three DP samples (lot #s 7, 9 and 4) and the “neat” HSA (lot #1).

FIG. 4B. Representative SEC traces for reconstituted products produced by vacuum drying (process-1), freeze-drying (process-2), and freeze-drying with pre- and post-filtration hold (process-3) (UV 280 nm detector). Inset shows graph of percent aggregation in the form of HSA polymer (left bars) and HSA dimer (right bars) in three DP samples (lot #s 7, 9 and 4) and the “neat” HSA (lot #1).

FIG. 4C. Representative SEC traces for reconstituted products produced by vacuum drying (process-1), freeze-drying (process-2), and freeze-drying with pre- and post-filtration hold (process-3) (fluorescence detector). Inset shows graph of percent aggregation in the form of HSA polymer (left bars), HSA oligomer (middle bars) and HSA dimer (right bars) in three DP samples (lot #s 7, 9 and 4) and the “neat” HSA (lot #1).

FIGS. 5A-5G. Total HSA aggregation in the DP lots manufactured with 3 production methods (vacuum dried (i.e., “Baseline, No holds, No Freezing”), freeze-dried (i.e., “Stress, Freezing Only”), and freeze-dried with pre-drying solution hold (i.e., “Stress, Holds at RT&Freezing”)), determined by SEC. Data of the neat HSA lots are also included. FIGS. 5A, 5C, and 5E show graphs for UV 220 nm detector, UV 280 nm detector, and fluorescence detector, respectively. FIGS. 5B, 5D, and 5F show the corresponding data values for UV 220 nm detector, UV 280 nm detector, and fluorescence detector, respectively. Data for certain lots are shown in FIG. 5G.

FIGS. 6A-6B. Particle counting results for three placebo lots produced using different manufacturing methods. FIG. 6A: Mean particle number per image is shown. Saline (left, n=1), baseline (i.e., vacuum dried, second from left, n=5, lot #6), stressed (holds at room temperature and freezing) (second from right, n=4, lot #11), stressed (freezing only) (right, n=4, lot #8). FIG. 6B: Particle size distributions (average results) are shown. Saline, baseline (i.e., vacuum dried, lot #6), stressed (holds at room temperature and freezing) (lot #11), stressed (freezing only) (lot #8).

FIG. 7A. pH and temperature changes during freezing and evaporative drying of the placebo solution. The blue arrow in this graph marks temperature of NaCl*2H₂O+water crystallization as detected in a separate WAXS experiment.

FIG. 7B. pH changes during evaporative drying of the placebo solution.

FIG. 8A. WAXS patterns during cooling of aqueous 0.9% NaCl/0.5% HSA placebo solution. Arrow: trace of NaCl*2H₂O.

FIG. 8B. WAXS patterns during cooling of aqueous 0.9% NaCl placebo solution.

FIG. 8C. SAXS patterns during cooling of 0.9% NaCl/0.5% HSA solution.

FIG. 9. Representative SEM images obtained at two magnifications. Dark areas represent HSA, and brighter areas correspond to NaCl or NaCl with HSA. left to right; vacuum dried (process-1), freeze-dried (process-2), and freeze-dried with pre-drying solution hold (process-3).

FIG. 10. X-ray diffraction patterns of dried placebo lots. Bottom curve: vacuum-dried (process-1, lot #6), top curve: freeze-dried with pre-drying hold (process-3, #11). Positions of the peaks of the placebo lots and NaCl (U.S.P., J. T. Baker, Batch 0000207378) are provided in the inset.

FIG. 11. Potency stability data tested at 25° C. storage condition. Top curve in each view: process-1, vacuum-dried. Bottom curve in each view: process-3, freeze-dried plus hold. Top view: merged view. Bottom view: split view.

### 5. DETAILED DESCRIPTION

The present invention provides solid compositions comprising BoNT/A and HSA with improved properties as well as manufacturing methods with an improved drying process for obtaining such solid compositions. Further benefits of the present disclosure will be apparent to one skilled in the art. The embodiments and aspects described in this disclosure are intended to illustrate the invention and should not be deemed to narrow the scope of the invention.

### 5.1. Definitions

The singular forms “a,” “an” and “the” include plural referents unless the context clearly dictates otherwise.

The term “and/or” as used in a phrase such as “A and/or B” herein is intended to mean “A and B”, “A or B”, “A” or “B”.

The terms “about” and “approximately” generally refer to a range of numbers that one of skill in the art would consider equivalent to the recited value (i.e., having the same function or result). In many instances, the terms “about” and “approximately” may include numbers that are rounded to the nearest significant figure. In specific embodiments, the terms “about” and “approximately” shall be construed so as to allow normal variation as judged by a person of ordinary skill in the art, such as, for example, a variation within 20% or 10% or 5%. In specific embodiments, the terms “about” and “approximately” encompass the exact value recited.

“Animal product free” (“APF”) or “substantially animal product free” encompasses, respectively, the absence or substantial absence of blood derived, blood pooled and other animal derived products or compounds. “Animal” excludes microorganisms, such as bacteria. Thus, an APF medium or process or a substantially APF medium or process within the scope of the present invention can include a botulinum toxin or a Clostridial botulinum bacterium. For example, an APF process or a substantially APF process means a process which is either substantially free or essentially free or entirely free of animal-derived proteins, such as immunoglobulins, meat digest, meat by-products and milk or dairy products or digests.

“Clostridium botulinum neurotoxin serotype A” or “BoNT/A” means a neurotoxin produced by Clostridium botulinum Type A strains. One such Clostridium botulinum Type A strain is the Type A-Hall strain, for example, the Type A-Hall (Allergan) strain. Zhang et al. (2003) Gene 315:21, incorporated herein by reference in its entirety. BoNT/A encompasses both a BoNT/A complex (e.g., the 300, 500, 760, and 900 kDa complexes) as well as pure BoNT/A toxin (i.e. the about 150 kDa neurotoxic molecule).

“BoNT/A complexes” means Clostridium botulinum serotype A neurotoxin complexes comprising a BoNT/A molecule (the neurotoxic component) and one or more hemagglutinin (HA) proteins and/or non-toxin non-hemagglutinin (NTNH) protein. The BoNT/A complexes can be in the forms of e.g., about 900 kDa, 760 kDa, 500 kDa or 300 kDa. In one embodiment, the BoNT/A complex is in the form of about 900 kDa, comprising an about 150 kDa BoNT/A molecule, hemagglutinin HA70, hemagglutinin HA34, hemagglutinin HA17, and nontoxic-nonhemagglutinin (NTNH) proteins. In one embodiment, the BoNT/A complex is a substantially complete form of the 900 kDa BoTN/A complex. In one embodiment, the BoNT/A complex is onabotulinumtoxinA.

“150 kDa Clostridium botulinum serotype A neurotoxin” or “150 kDa BoNT/A” means a neurotoxin of approximately 150 kDa made from a culture of Clostridium botulinum type A strain (e.g., the Hall strain of Clostridium botulinum). The preferred sequences of 150 kDa botulinum toxin type A (BoNT/A) used in the context of the present disclosure are shown in Table 1. For example, in one embodiment, the 150 kDa BoNT/A used in the context of the present disclosure comprises (e.g., consists of) a light chain having an amino acid sequence set forth in SEQ ID NO. 2 and a heavy chain having an amino acid sequence set forth in SEQ ID NO. 3, with disulfide bridges located between positions 429 and 453 and between positions 1234 and 1279.

“BoNT/A composition” refers to any composition comprising BoNT/A and encompasses both solid compositions and liquid compositions. In certain embodiments, a BoNT/A composition (e.g., a solid composition or liquid composition) described herein is a pharmaceutical composition. In specific embodiments, a BoNT/A composition (e.g., a solid composition or liquid composition) described herein is a drug product (i.e., a finished dosage form). In a preferred embodiment, a BoNT/A composition described herein is in the form of powder (e.g., vacuum-dried powder).

The term “carrier” used in connection with a pharmaceutical excipient refers to any and all solvents, dispersion media, preservatives, coatings, isotonic and absorption delaying agents, and the like, that are compatible with pharmaceutical administration.

The term “patient”, “subject”, “individual” and the like refers to humans.

“Pharmaceutical composition” means a formulation in which an active ingredient can be a BoNT/A. The word “formulation” means that there is at least one additional ingredient (such as, for example and not limited to, an albumin (such as a human serum albumin (HSA) or a recombinant human albumin) and/or sodium chloride) in the pharmaceutical composition in addition to a BoNT/A active ingredient. The human serum albumin excipient can be derived from human plasma or recombinantly made. A pharmaceutical composition is therefore a formulation which is suitable for diagnostic, therapeutic and/or cosmetic administration (e.g., by intramuscular or subcutaneous injection or by insertion of a depot or implant) to a subject, such as a human patient. In one embodiment, the active ingredient is onabotulinumtoxinA. Exemplary methods for formulating a BoNT/A active ingredient pharmaceutical composition are disclosed in U.S. Patent Application Publication No. 2003/0118598, filed Nov. 5, 2002, herein incorporated by reference in its entirety. In a preferred embodiment, a pharmaceutical composition described herein is in a dried form (e.g., a vacuum-dried form). The pharmaceutical compositions can be vacuum-dried and suitable for administration by injection either subcutaneously or intramuscularly upon reconstitution with normal saline, comprising 900 kDa BoNT/A, human serum albumin (HSA), and sodium chloride. Preferably such pharmaceutical compositions comprise 0.5 mg of HSA and 0.9 mg of sodium chloride per 100 Units of BoNT/A. Most preferably such pharmaceutical compositions comprise 50, 100 or 200 Units of BoNT/A.

“Unit” or “U” refers to the LD₅₀ dose or the dose determined by a cell-based potency assay (CBPA). The LD₅₀ dose is defined as the amount of BoNT/A that killed 50% of the mice injected with the BoNT/A. The CBPA dose is determined as described in U.S. Pat. Nos. 8,618,261; 8,198,034; 9,249,216; 10,703,806; 11,261,240 and 11,332,518; the assay details of which are incorporated by reference herein.

Unless the context requires otherwise, the terms “comprise,” “comprises,” and “comprising” are used on the basis and clear understanding that they are to be interpreted inclusively, rather than exclusively, such that they indicate the inclusion of the recited feature but without excluding one or more other such features. However, it is understood that wherever aspects and embodiments are described herein with the language “comprise” (or “comprises” or “comprising”), otherwise analogous aspects described in terms of “consist of” (or “consists of” or “consisting of”) and/or “consist essentially of” (or “consists essentially of” or “consisting essentially of”) are also provided.

### 5.2. Clostridium Botulinum Neurotoxin Serotype A (BoNT/A)

Clostridial bacterium can produce botulinum toxin type A complexes in various forms, which include, and are not limited to, 900 kDa, 760 kDa, 500 kDa, and 300 kDa complexes (approximate molecular weights).

In one embodiment, the BoNT/A described herein is present as a 900 kDa BoNT/A complex. In one embodiment, the BoNT/A described herein is present as a 900 kDa BoNT/A complex formed by the 150 kDa BoNT/A molecule and hemagglutinin HA70, hemagglutinin HA34, hemagglutinin HA17, and nontoxic-nonhemagglutinin (NTNH) proteins. In a specific embodiment, the BoNT/A described herein (e.g., the 900 kDa BoNT/A complex) is produced in a Clostridium botulinum type A strain. In a specific embodiment, the BoNT/A described herein (e.g., the 900 kDa BoNT/A complex) is produced in a Clostridium botulinum type A Hall strain. In a preferred embodiment, the BoNT/A described herein is onabotulinumtoxinA.

In one embodiment, the 150 kDa BoNT/A molecule that can be used in the context of the present disclosure has a sequence shown in Table 1. In one embodiment, the 150 kDa BoNT/A molecule comprises a light chain (LC: residues 2-438, about 50 kDa) and a heavy chain (HC: residues 449-1296, about 100 kDa). Residues 439-448 are the nicking site and are italicized.

TABLE 1
Preferred sequences of BoNT/A molecules.

```
SEQ ID NO. 1:
amino acid sequence of the 150 kDa BoNT/A molecule
MPFVNKQFNY KDPVNGVDIA YIKIPNAGQM QPVKAFKIHN KIWVIPERDT 50
FTNPEEGDLN PPPEAKQVPV SYYDSTYLST DNEKDNYLKG VTKLFERIYS 100
TDLGRMLLTS IVRGIPFWGG STIDTELKVI DTNCINVIQP DGSYRSEELN 150
LVIIGPSADI IQFECKSFGH EVLNLTRNGY GSTQYIRFSP DFTFGFEESL 200
EVDTNPLLGA GKFATDPAVT LAHELIHAGH RLYGIAINPN RVFKVNTNAY 250
YEMSGLEVSF EELRTFGGHD AKFIDSLQEN EFRLYYYNKF KDIASTLNKA 300
KSIVGTTASL QYMKNVFKEK YLLSEDTSGK FSVDKLKFDK LYKMLTEIYT 350
EDNFVKFFKV LNRKTYLNFD KAVFKINIVP KVNYTIYDGF NLRNTNLAAN 400
FNGQNTEINN MNFTKLKNFT GLFEFYKLLC VRGIITSKTK SLDKGYNK
                                                                                                        AL 450
QYYLTFNFDN EPENISIENL SSDIIGQLEL MPNIERFPNG KKYELDKYTM 550
FHYLRAQEFE HGKSRIALTN SVNEALLNPS RVYTFFSSDY VKKVNKATEA 600
AMFLGWVEQL VYDFTDETSE VSTTDKIADI TIIIPYIGPA LNIGNMLYKD 650
DFVGALIFSG AVILLEFIPE IAIPVLGTFA LVSYIANKVL TVQTIDNALS 700
KRNEKWDEVY KYIVTNWLAK VNTQIDLIRK KMKEALENQA EATKAIINYQ 750
YNQYTEEEKN NINFNIDDLS SKLNESINKA MININKFLNQ CSVSYLMNSM 800
IPYGVKRLED FDASLKDALL KYIYDNRGTL IGQVDRLKDK VNNTLSTDIP 850
FQLSKYVDNQ RLLSTFTEYI KNIINTSILN LRYESNHLID LSRYASKINI 900
GSKVNFDPID KNQIQLFNLE SSKIEVILKN AIVYNSMYEN FSTSFWIRIP 950
KYFNSISLNN EYTIINCMEN NSGWKVSLNY GEIIWTLQDT QEIKQRVVFK 1000
YSQMINISDY INRWIFVTIT NNRLNNSKIY INGRLIDQKP ISNLGNIHAS 1050
NNIMFKLDGC RDTHRYIWIK YFNLFDKELN EKEIKDLYDN QSNSGILKDF 1100
WGDYLQYDKP YYMLNLYDPN KYVDVNNVGI RGYMYLKGPR GSVMTTNIYL 1150
NSSLYRGTKF IIKKYASGNK DNIVRNNDRV YINVVVKNKE YRLATNASQA 1200
GVEKILSALE IPDVGNLSQV VVMKSKNDQG ITNKCKMNLQ DNNGNDIGFI 1250
GFHQFNNIAK LVASNWYNRQ IERSSRTLGC SWEFIPVDDG WGERPL 1296
SEQ ID NO. 2:
amino acid sequence of the light chain (LC) of BoNT/A molecule
  PFVNKQFNY KDPVNGVDIA YIKIPNAGQM QPVKAFKIHN KIWVIPERDT 50
FTNPEEGDLN PPPEAKQVPV SYYDSTYLST DNEKDNYLKG VTKLFERIYS 100
TDLGRMLLTS IVRGIPFWGG STIDTELKVI DTNCINVIQP DGSYRSEELN 150
LVIIGPSADI IQFECKSFGH EVLNLTRNGY GSTQYIRFSP DFTFGFEESL 200
EVDTNPLLGA GKFATDPAVT LAHELIHAGH RLYGIAINPN RVFKVNTNAY 250
YEMSGLEVSF EELRTFGGHD AKFIDSLQEN EFRLYYYNKF KDIASTLNKA 300
KSIVGTTASL QYMKNVFKEK YLLSEDTSGK FSVDKLKFDK LYKMLTEIYT 350
EDNFVKFFKV LNRKTYLNFD KAVFKINIVP KVNYTIYDGF NLRNTNLAAN 400
                                                                                                        AL 450
NDLCIKVNNW DLFFSPSEDN FINDLNKGEE ITSDTNIEAA EENISLDLIQ 500
FNGQNTEINN MNFTKLKNFT GLFEFYKLLC VRGIITSK
SEQ ID NO. 3:
amino acid sequence of the heavy chain (HC) of BoNT/A molecule
NDLCIKVNNW DLFFSPSEDN FINDLNKGEE ITSDTNIEAA EENISLDLIQ 500
QYYLTFNFDN EPENISIENL SSDIIGQLEL MPNIERFPNG KKYELDKYTM 550
FHYLRAQEFE HGKSRIALTN SVNEALLNPS RVYTFFSSDY VKKVNKATEA 600
AMFLGWVEQL VYDFTDETSE VSTTDKIADI TIIIPYIGPA LNIGNMLYKD 650
DFVGALIFSG AVILLEFIPE IAIPVLGTFA LVSYIANKVL TVQTIDNALS 700
KRNEKWDEVY KYIVTNWLAK VNTQIDLIRK KMKEALENQA EATKAIINYQ 750
YNQYTEEEKN NINFNIDDLS SKLNESINKA MININKFLNQ CSVSYLMNSM 800
IPYGVKRLED FDASLKDALL KYIYDNRGTL IGQVDRLKDK VNNTLSTDIP 850
FQLSKYVDNQ RLLSTFTEYI KNIINTSILN LRYESNHLID LSRYASKINI 900
GSKVNFDPID KNQIQLFNLE SSKIEVILKN AIVYNSMYEN FSTSFWIRIP 950
KYFNSISLNN EYTIINCMEN NSGWKVSLNY GEIIWTLQDT QEIKQRVVFK 1000
YSQMINISDY INRWIFVTIT NNRLNNSKIY INGRLIDQKP ISNLGNIHAS 1050
NNIMFKLDGC RDTHRYIWIK YFNLFDKELN EKEIKDLYDN QSNSGILKDF 1100
WGDYLQYDKP YYMLNLYDPN KYVDVNNVGI RGYMYLKGPR GSVMTTNIYL 1150
NSSLYRGTKF IIKKYASGNK DNIVRNNDRV YINVVVKNKE YRLATNASQA 1200
GVEKILSALE IPDVGNLSQV VVMKSKNDQG ITNKCKMNLQ DNNGNDIGFI 1250
GFHQFNNIAK LVASNWYNRQ IERSSRTLGC SWEFIPVDDG WGERPL 1296
```

### 5.3. Production of BoNT/A and Production of BoNT/A Compositions

Botulinum toxin type A has been approved by the U.S. Food and Drug Administration (FDA) for the treatment of essential blepharospasm, strabismus and hemifacial spasm in patients over the age of twelve, cervical dystonia, glabellar line (facial) wrinkles and for treating hyperhydrosis. A commercially available botulinum toxin type A containing pharmaceutical composition is sold under the trademark BOTOX® (onabotulinumtoxinA), available commercially from Allergan, an Abb Vie company, North Chicago, Illinois, USA. BOTOX® contains a purified 900 kDa botulinum toxin type A complex, human serum albumin, and sodium chloride packaged in sterile, vacuum-dried form. The botulinum toxin type A complex in BOTOX® is made from a culture of the Hall strain of Clostridium botulinum grown in a medium containing N-Z amine casein and yeast extract (i.e., non-APF process) and purified from the culture solution by a series of precipitation (including acid precipitation) steps to a crystalline complex consisting of the active high molecular weight toxin protein and an associated hemagglutinin protein. The crystalline complex is re-dissolved in a solution containing saline and albumin and sterile filtered using a gamma-irradiated filter (0.2 microns) prior to vacuum-drying. BOTOX® can be reconstituted with sterile, non-preserved saline prior to intramuscular injection. Each 100 unit vial of BOTOX® consists of about 5 ng of purified botulinum toxin type A complex, 0.5 mg human serum albumin, and 0.9 mg sodium chloride, vacuum-dried form and intended for reconstitution with sterile normal saline without a preservative (0.9% sodium chloride injection).

In general, the production of a BoNT/A pharmaceutical composition involves first making the BoNT/A drug substance and then compounding the BoNT/A drug substance with excipient(s). A drying step is often employed at the end of the production process to produce a dried form of the pharmaceutical composition for easier storage and/or transportation. This disclosure describes steps and measures that may be taken to ensure that the drug product does not undergo freezing during the drying process, as freezing during drying unexpectedly results in a significant increase in the amount of HSA aggregation in BoNT/A compositions.

A number of steps are required to make BoNT/A drug substances (i.e., to obtain purified BoNT/A). A first step can be to culture a Clostridial bacteria (e.g., the Hall strain of Clostridium botulinum), typically on agar plates, in an environment conducive to bacterial growth, such as in a warm anaerobic atmosphere. The culture step allows Clostridial colonies with desirable morphology and other characteristics to be obtained. The culture step can also be performed with bacteria from an animal product free working cell back. In a second step, selected cultured Clostridial colonies can be fermented in a suitable medium. After a certain period of fermentation the Clostridial bacteria typically lyse and release Clostridial toxin (e.g., BoNT/A) into the medium. Thirdly, the toxin can be purified from the culture medium to obtain a bulk or raw BoNT/A toxin drug substance. Preferably, the BoNT/A toxin drug substance will not have been subjected to precipitation (e.g., precipitation with cold ethanol, hydrochloric acid, and/or ammonium sulfate), e.g., during purification. In certain aspects of the invention, BoNT/A toxin drug substance is purified by column chromatography including a hydrophobic interaction chromatography (HIC) column. In certain aspects of the invention, when multiple chromatography columns are used to purify the BoNT/A, the toxin is purified using a process wherein a HIC column is used prior to all other chromatography columns. It is preferable that BoNT/A drug substance is produced by a process that qualifies for use under the Good Manufacturing Practice regulations promulgated by the U.S. Food and Drug Administration.

In some embodiments, the BoNT/A drug substance is obtained using a substantially, essentially or entirely animal protein free (APF) process. The process can comprise APF or substantially APF culture and fermentation processes. An APF or substantially APF chromatographic system and process can be used to purify a clarified culture of Clostridium botulinum obtained from the APF or substantially APF culture and fermentation processes. In some embodiments, the chromatography-based purification process is as disclosed in U.S. Pat. No. 8,129,139, which is incorporated by reference herein in its entirety.

The BoNT/A drug substance can be produced by exemplary methods as described in Example 1 of U.S. Pat. No. 8,129,139, Example 2 of U.S. Pat. No. 8,129,139, or Example 1 of U.S. Pat. No. 9,469,849 (which is incorporated by reference herein in its entirety), or using one or more steps as described in one or more of these examples.

In certain embodiments, the BoNT/A drug substance is produced by a process that comprises one or more steps of column chromatography (e.g., one or more steps of column chromatography performed during purification of the BoNT/A drug substance).

In preferred embodiments, the one or more steps of column chromatography (e.g., performed during purification of the BoNT/A drug substance) comprise hydrophobic interaction chromatography, anion exchange chromatography, and/or cation exchange chromatography, wherein preferably hydrophobic interaction chromatography is performed before anion exchange chromatography and/or cation exchange chromatography.

In certain embodiments, the BoNT/A drug substance is produced by a process that does not comprise a step of precipitation with cold ethanol, hydrochloric acid, or ammonia sulfate (e.g., during purification of the BoNT/A drug substance).

In certain embodiments, the BoNT/A drug substance is produced by a process that does not involve using a protease inhibitor. In certain embodiments, the BoNT/A drug substance is produced by a process that does not involve using benzamidine hydrocholoride.

After the BoNT/A drug substance is produced, it can be stabilized in a suitable solution. The BoNT/A drug substance can then be compounded with one or more excipients (e.g., human serum albumin, such as recombinant human serum albumin, and sodium chloride) and further sterile filtered to make a pharmaceutical composition suitable for administration to a human. The pharmaceutical compositions can be made into solid forms (e.g., as powder) by drying (e.g., vacuum-drying). Solid pharmaceutical compositions can be stored and reconstituted prior to injection. The BoNT/A pharmaceutical compositions described herein can comprise a 900 kDa BoNT/A complex as an active pharmaceutical ingredient. The pharmaceutical composition can also include one or more excipients, buffers, carriers, stabilizers, preservatives and/or bulking agents. Such pharmaceutical compositions are preferably chemically and physically stable such that the BoNT/A active pharmaceutical ingredient remains suitable for use as a pharmaceutical product following storage. BoNT/A products may be stored at room temperature, in refrigerated conditions, or below 0° C. It is preferable that the BoNT/A remains stable during storage for at least about 12 months, more preferably at least about 18 months.

In various aspects and embodiments, a solid BoNT/A composition described herein is dried from a solution comprising BoNT/A drug substance (for example, BoNT/A drug substance produced by a method described herein) and HSA. Specifically, the present invention provides a method of producing a solid composition comprising BoNT/A and HSA, said method comprising a step of drying a solution comprising BoNT/A and HSA to produce said solid composition, wherein the solution does not undergo freezing during the drying step. In specific embodiments, the method produces a solid composition described herein.

Whether a solution described herein (or a reference solution described herein, as the case may be) undergoes freezing during drying may be determined by monitoring its temperature during drying (for example, as described in Example 1). The monitoring may be done by inserting a temperature probe into the solution (or reference solution, as the case may be). The monitoring may be done by using a Pirani gauge or an Ellab temperature probe. If multiple vials of the solution (or reference solution, as the case may be) are dried together (e.g., in the same drying chamber or on the same drying tray), then the monitoring may be done for one or more (e.g., one or two) representative vials and do not have to be done for all of the vials. In certain embodiments, a drying process during which a solution does not undergo freezing comprises maintaining the temperature of the solution at above its freezing point during drying. In certain embodiments, a drying process during which a solution does not undergo freezing comprises maintaining the temperature of the solution at above 0° C. during drying.

Whether a solution described herein (or a reference solution described herein, as the case may be) undergoes freezing during drying may also be determined by testing the dried solid composition (or the dried reference solid composition, as the case may be) using scanning electron microscope (SEM) (for example, as described in Example 1). If multiple vials of the solution (or reference solution, as the case may be) are dried together (e.g., in the same drying chamber or on the same drying tray), then the testing may be done for one or more (e.g., one or two) representative vials of the dried solid compositions and do not have to be done for all of the vials. In certain embodiments, the absence of pores in a dried solid composition indicates that it is dried from a solution that has not undergone freezing during drying.

In various embodiments and aspects, the step of drying is at least partially manually controlled. In various embodiments and aspects, the step of drying is completely manually controlled. In various embodiments and aspects, the step of drying is at least partially digitally controlled. In various embodiments and aspects, the step of drying is completely digitally controlled.

In various embodiments and aspects, the step of drying is vacuum drying, spray drying, convective drying, microwave drying, or a combination thereof.

In various embodiments and aspects, the step of drying is vacuum drying. To avoid freezing of the drug product solution, a controlled depressurization/evacuation phase may be performed. Measures that can be taken during the controlled depressurization/evacuation phase to help prevent freezing include: using a slower chamber depressurization rate, conducting depressurization in staggered steps, monitoring the drug product solution (for example, to ensure that it's certain degrees above the freezing point), and/or using a high shelf temperature (for example, setting the shelf temperature to about 20° C. to 25° C.). The exact chamber depressurization rates and steps to be employed may depend on the drying equipment, the chamber size, the condenser capacity, the chamber load, and/or the vacuum pump performance, etc., and may be determined by a person of ordinary skill in the art and may involve manual adjustment. For example, in certain embodiments, the vacuum drying step may comprise a stepwise evacuating step, which can be performed in such a way that the temperature of a solution described herein is allowed to decline under evacuation until it reaches a temperature that is still above the freezing point of the solution and preferably close to the freezing point of the solution (e.g., about 2° C. to 5° C. above the freezing point of the solution), and then the evacuation is stopped until the temperature of the solution goes back up to a higher temperature (e.g., about 20° C. to 25° C.), and then the evacuation is resumed. The foregoing step can be repeated for once or more than once, while the exact temperature at which the evacuation is stopped or resumed does not need to be the same for each repeat. In certain other embodiments, the vacuum drying step may comprise a slow evacuating step.

### 5.4. BoNT/A Compositions

The present disclosure provides solid compositions comprising Clostridium botulinum neurotoxin serotype A (BoNT/A) and human serum albumin (HSA), wherein the solid compositions comprise a low amount of HSA aggregates.

HSA contained in a BoNT/A composition can exist in the form of HSA monomer or HSA aggregates. There can be two types of HSA aggregates, i.e., soluble HSA aggregates and insoluble HSA aggregates. Soluble HSA aggregates can exist in the form of HSA polymer, HSA oligomer, HSA dimer, or a mixture thereof. In certain embodiments, soluble HSA polymer, soluble HSA oligomer, soluble HSA dimer, and soluble HSA monomer can be separated by size exclusion chromatography (SEC). For example, peaks on an SEC chromatogram can be assigned to soluble HSA polymer, soluble HSA oligomer, soluble HSA dimer, and soluble HSA monomer, respectively, based on retention times obtained when running a standard control (e.g., a gel filtration standard consisting of bovine thyroglobulin (MW 670 kDa), bovine γ-globulin (MW 158 kDa), chicken albumin (MW 44 kDa), horse myoglobulin (MW 17 kDa), and Vitamin B12 (MW 1350 Da), such as the Biorad gel filtration standard (Cat 151-1901)) on the same column. In a specific embodiment, peaks on an SEC chromatogram can be assigned to soluble HSA polymer, soluble HSA oligomer, soluble HSA dimer, and soluble HSA monomer, respectively, as described in Example 1.

Suitable methods for measuring the level of soluble and/or insoluble HSA aggregates include, but are not limited to, dynamic light scattering (DLS), gel electrophoresis (e.g., sodium dodecyl-sulfate polyacrylamide gel electrophoresis (SDS-PAGE)), transmission electron microscopy (TEM), ultracentrifugation (e.g., analytical ultracentrifugation (AUC), such as sedimentation velocity analytical ultracentrifugation (SV-AUC)), asymmetrical fast field flow fractionation (FFF or AF4), and turbidity measurements.

Suitable methods for measuring the level of HSA aggregates, polymer, oligomer, or dimer in soluble HSA include, but are not limited to, HPLC (e.g., SEC), DLS, gel electrophoresis (e.g., SDS-PAGE), ultracentrifugation (e.g., AUC, such as SV-AUC), asymmetrical fast field flow fractionation (FFF or AF4), and turbidity measurements.

Suitable methods for measuring the level of insoluble HSA aggregates include, but are not limited to, DLS, gel electrophoresis (e.g., SDS-PAGE), transmission electron microscopy (TEM), ultracentrifugation (e.g., AUC, such as SV-AUC), asymmetrical fast field flow fractionation (FFF or AF4), microscopy (e.g., optical microscopy), micro-flow imaging (MFI), turbidity measurements, light obscuration (e.g., by HIAC (high accuracy liquid particle counter)), and detection of subvisible particles (such that the percentage of insoluble HSA aggregates is or is represented by the percentage of subvisible particles) (for example, a noninvasive subvisible particle detection method (e.g., a noninvasive subvisible particle detection method described in Example 1 or described in U.S. Pat. No. 10,132,736 B2, which is incorporated by reference herein in its entirety, optical microscopy, MFI, or light obscuration (e.g., by HIAC)).

In certain embodiments, the percentage of HSA aggregates or the percentage of a type of HSA aggregates in a solid composition described herein is determined after at least a portion of the solid composition is reconstituted. Reconstitution of the solid composition (or a portion thereof) can be performed by mixing the solid composition (or a portion thereof) with liquid (for example, water or saline). In specific embodiments, the reconstituted composition comprises about 0.01 mg/ml to about 100 mg/ml, about 0.1 mg/ml to about 10 mg/ml, about 0.25 mg/ml to about 2 mg/ml, about 0.25 mg/ml, about 0.5 mg/ml, about 1 mg/ml, or about 2 mg/ml HSA. In certain embodiments, the percentage of HSA aggregates or the percentage of a type of HSA aggregates in a solid composition described herein is determined using SEC after at least a portion of the solid composition is reconstituted. Detectors that may be used to detect soluble HSA aggregates (for example, when using SEC) include, but are not limited to, UV detectors (e.g., UV detectors with a wavelength of about 220 nm, UV detectors with a wavelength of about 280 nm, UV detectors with a wavelength of about 254 nm, and UV detectors with a wavelength of between 220 nm and 280 nm) and fluorescence detectors (e.g., fluorescence detectors with an excitation wavelength of about 280 nm and an emission wavelength of about 350 nm).

The present invention provides solid compositions comprising BoNT/A and HSA, wherein the solid compositions comprise a low percentage of HSA aggregates, including HSA polymer, oligomer, and/or dimer. As shown in Example 1, a BoNT/A composition dried without freezing not only has an overall low percentage of HSA aggregates, but also has low percentages of each of HSA polymer, oligomer, and dimer.

In specific embodiments, the percentage of HSA aggregates, the percentage of HSA polymer, the percentage of HSA oligomer, and/or the percentage of HSA dimer, in the soluble HSA contained in the solid composition is determined using SEC by a 220 nm wavelength UV detector after at least a portion of the solid composition is reconstituted. In specific embodiments, the percentage of HSA aggregates, the percentage of HSA polymer, the percentage of HSA oligomer, and/or the percentage of HSA dimer, in the soluble HSA contained in the solid composition is determined using SEC by a 280 nm wavelength UV detector after at least a portion of the solid composition is reconstituted. In specific embodiments, the percentage of HSA aggregates, the percentage of HSA polymer, the percentage of HSA oligomer, and/or the percentage of HSA dimer, in the soluble HSA contained in the solid composition is determined using SEC by a fluorescence detector with an excitation wavelength of 280 nm and an emission wavelength of 350 nm by a fluorescence detector with an excitation wavelength of 280 nm and an emission wavelength of 350 nm after at least a portion of the solid composition is reconstituted.

In one aspect, provided herein is a solid composition comprising BoNT/A and HSA, wherein the HSA comprises HSA monomer and HSA aggregates, wherein at least a portion of the HSA is in the form of soluble HSA when the solid composition is reconstituted in an aqueous medium, and wherein less than 34% of soluble HSA contained in the solid composition are HSA aggregates. It shall be understood in this disclosure that the soluble HSA aggregates described herein include all types of soluble HSA aggregates (i.e., soluble HSA dimer, soluble HSA polymer, and soluble HSA oligomer). In specific embodiments, less than 33%, less than 32%, less than 31%, less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of soluble HSA contained in the solid composition are HSA aggregates. In specific embodiments, about 1% to about 4%, about 4% to about 6%, about 6% to about 10%, about 4% to about 10%, about 10% to about 12%, about 10% to about 15%, about 15% to about 18%, about 15% to about 20%, about 20% to about 25%, about 25% to about 30%, or about 30% to about 33% of soluble HSA contained in the solid composition are HSA aggregates.

In specific embodiments, less than 19% of soluble HSA contained in the solid composition are HSA aggregates, as determined using SEC by a 220 nm wavelength UV detector after at least a portion of the solid composition is reconstituted. In specific embodiments, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of soluble HSA contained in the solid composition are HSA aggregates, as determined using SEC by a 220 nm wavelength UV detector after at least a portion of the solid composition is reconstituted. In specific embodiments, about 1% to about 4%, about 4% to about 6%, about 6% to about 10%, about 4% to about 10%, about 10% to about 12%, about 10% to about 15%, or about 15% to about 18% of soluble HSA contained in the solid composition are HSA aggregates, as determined using SEC by a 220 nm wavelength UV detector after at least a portion of the solid composition is reconstituted.

In specific embodiments, less than 34% of soluble HSA contained in the solid composition are HSA aggregates, as determined using SEC by a 280 nm wavelength UV detector after at least a portion of the solid composition is reconstituted. In specific embodiments, less than 33%, less than 32%, less than 31%, less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of soluble HSA contained in the solid composition are HSA aggregates, as determined using SEC by a 280 nm wavelength UV detector after at least a portion of the solid composition is reconstituted. In specific embodiments, about 1% to about 4%, about 4% to about 6%, about 6% to about 10%, about 4% to about 10%, about 10% to about 12%, about 10% to about 15%, about 15% to about 18%, about 15% to about 20%, about 20% to about 25%, about 25% to about 30%, or about 30% to about 33% of soluble HSA contained in the solid composition are HSA aggregates, as determined using SEC by a 280 nm wavelength UV detector after at least a portion of the solid composition is reconstituted.

In specific embodiments, less than 20% of soluble HSA contained in the solid composition are HSA aggregates, as determined using SEC by a fluorescence detector with an excitation wavelength of 280 nm and an emission wavelength of 350 nm by a fluorescence detector with an excitation wavelength of 280 nm and an emission wavelength of 350 nm after at least a portion of the solid composition is reconstituted. In specific embodiments, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of soluble HSA contained in the solid composition are HSA aggregates, as determined using SEC by a fluorescence detector with an excitation wavelength of 280 nm and an emission wavelength of 350 nm by a fluorescence detector with an excitation wavelength of 280 nm and an emission wavelength of 350 nm after at least a portion of the solid composition is reconstituted. In specific embodiments, about 1% to about 4%, about 4% to about 6%, about 6% to about 10%, about 4% to about 10%, about 10% to about 12%, about 10% to about 15%, or about 15% to about 18% of soluble HSA contained in the solid composition are HSA aggregates, as determined using SEC by a fluorescence detector with an excitation wavelength of 280 nm and an emission wavelength of 350 nm after at least a portion of the solid composition is reconstituted.

In another aspect, provided herein is a solid composition comprising BoNT/A and HSA, wherein at least a portion of the HSA is in the form of soluble HSA when the solid composition is reconstituted in an aqueous medium, wherein less than 30% of soluble HSA contained in the solid composition is HSA polymer. In specific embodiments, less than 29%, less than 28%, less than 27%, less than 26%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of soluble HSA contained in the solid composition is HSA polymer. In specific embodiments, about 1% to about 4%, about 4% to about 6%, about 6% to about 10%, about 4% to about 10%, about 10% to about 12%, about 10% to about 15%, about 15% to about 18%, about 15% to about 20%, about 20% to about 25%, or about 25% to about 29% of soluble HSA contained in the solid composition is HSA polymer.

In specific embodiments, less than 13% of soluble HSA contained in the solid composition is HSA polymer, as determined using SEC by a 220 nm wavelength UV detector after at least a portion of the solid composition is reconstituted. In specific embodiments, less than 12%, less than 11%, less than 10%, less than 9%, less than 8%, less than 7%, less than 6%, less than 5%, less than 4%, less than 3%, less than 2%, or less than 1% of soluble HSA contained in the solid composition is HSA polymer, as determined using SEC by a 220 nm wavelength UV detector after at least a portion of the solid composition is reconstituted. In specific embodiments, about 1% to about 4%, about 4% to about 6%, about 6% to about 10%, about 4% to about 10%, or about 10% to about 12% of soluble HSA contained in the solid composition is HSA polymer, as determined using SEC by a 220 nm wavelength UV detector after at least a portion of the solid composition is reconstituted.

In specific embodiments, less than 30% of soluble HSA contained in the solid composition is HSA polymer, as determined using SEC by a 280 nm wavelength UV detector after at least a portion of the solid composition is reconstituted. In specific embodiments, less than 29%, less than 28%, less than 27%, less than 26%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of soluble HSA contained in the solid composition is HSA polymer, as determined using SEC by a 280 nm wavelength UV detector after at least a portion of the solid composition is reconstituted. In specific embodiments, about 1% to about 4%, about 4% to about 6%, about 6% to about 10%, about 4% to about 10%, about 10% to about 12%, about 10% to about 15%, about 15% to about 18%, about 15% to about 20%, about 20% to about 25%, or about 25% to about 29% of soluble HSA contained in the solid composition is HSA polymer, as determined using SEC by a 280 nm wavelength UV detector after at least a portion of the solid composition is reconstituted.

In specific embodiments, less than 13% of soluble HSA contained in the solid composition is HSA polymer, as determined using SEC by a fluorescence detector with an excitation wavelength of 280 nm and an emission wavelength of 350 nm after at least a portion of the solid composition is reconstituted. In specific embodiments, less than 12%, less than 11%, less than 10%, less than 9%, less than 8%, less than 7%, less than 6%, less than 5%, less than 4%, less than 3%, less than 2%, or less than 1% of soluble HSA contained in the solid composition is HSA polymer, as determined using SEC by a fluorescence detector with an excitation wavelength of 280 nm and an emission wavelength of 350 nm after at least a portion of the solid composition is reconstituted. In specific embodiments, about 1% to about 4%, about 4% to about 6%, about 6% to about 10%, about 4% to about 10%, or about 10% to about 12% of soluble HSA contained in the solid composition is HSA polymer, as determined using SEC by a fluorescence detector with an excitation wavelength of 280 nm and an emission wavelength of 350 nm after at least a portion of the solid composition is reconstituted.

In another aspect, provided herein is a solid composition comprising BoNT/A and HSA, wherein at least a portion of the HSA is in the form of soluble HSA when the solid composition is reconstituted in an aqueous medium, wherein less than 0.8% of soluble HSA contained in the solid composition is HSA oligomer. In specific embodiments, less than 0.7%, less than 0.6%, less than 0.5%, less than 0.4%, less than 0.3%, less than 0.2%, or less than 0.1% of soluble HSA contained in the solid composition is HSA oligomer. In specific embodiments, about 0.1% to about 0.2%, about 0.2% to about 0.3%, about 0.3% to about 0.4%, about 0.4% to about 0.5%, about 0.5% to about 0.6%, or about 0.6% to about 0.7% of soluble HSA contained in the solid composition is HSA oligomer.

In specific embodiments, less than 0.8% of soluble HSA contained in the solid composition is HSA oligomer, as determined using SEC by a 220 nm wavelength UV detector after at least a portion of the solid composition is reconstituted. In specific embodiments, less than 0.7%, less than 0.6%, less than 0.5%, less than 0.4%, less than 0.3%, less than 0.2%, or less than 0.1% of soluble HSA contained in the solid composition is HSA oligomer, as determined using SEC by a 220 nm wavelength UV detector after at least a portion of the solid composition is reconstituted. In specific embodiments, about 0.1% to about 0.2%, about 0.2% to about 0.3%, about 0.3% to about 0.4%, about 0.4% to about 0.5%, about 0.5% to about 0.6%, or about 0.6% to about 0.7% of soluble HSA contained in the solid composition is HSA oligomer, as determined using SEC by a 220 nm wavelength UV detector after at least a portion of the solid composition is reconstituted.

In specific embodiments, less than 0.8% of soluble HSA contained in the solid composition is HSA oligomer, as determined using SEC by a 280 nm wavelength UV detector after at least a portion of the solid composition is reconstituted. In specific embodiments, less than 0.7%, less than 0.6%, less than 0.5%, less than 0.4%, less than 0.3%, less than 0.2%, or less than 0.1% of soluble HSA contained in the solid composition is HSA oligomer, as determined using SEC by a 280 nm wavelength UV detector after at least a portion of the solid composition is reconstituted. In specific embodiments, about 0.1% to about 0.2%, about 0.2% to about 0.3%, about 0.3% to about 0.4%, about 0.4% to about 0.5%, about 0.5% to about 0.6%, or about 0.6% to about 0.7% of soluble HSA contained in the solid composition is HSA oligomer, as determined using SEC by a 280 nm wavelength UV detector after at least a portion of the solid composition is reconstituted.

In specific embodiments, less than 0.8% of soluble HSA contained in the solid composition is HSA oligomer, as determined using SEC by a fluorescence detector with an excitation wavelength of 280 nm and an emission wavelength of 350 nm after at least a portion of the solid composition is reconstituted. In specific embodiments, less than 0.7%, less than 0.6%, less than 0.5%, less than 0.4%, less than 0.3%, less than 0.2%, or less than 0.1% of soluble HSA contained in the solid composition is HSA oligomer, as determined using SEC by a fluorescence detector with an excitation wavelength of 280 nm and an emission wavelength of 350 nm after at least a portion of the solid composition is reconstituted. In specific embodiments, about 0.1% to about 0.2%, about 0.2% to about 0.3%, about 0.3% to about 0.4%, about 0.4% to about 0.5%, about 0.5% to about 0.6%, or about 0.6% to about 0.7% of soluble HSA contained in the solid composition is HSA oligomer, as determined using SEC by a fluorescence detector with an excitation wavelength of 280 nm and an emission wavelength of 350 nm after at least a portion of the solid composition is reconstituted.

In another aspect, provided herein is a solid composition comprising BoNT/A and HSA, wherein at least a portion of the HSA is in the form of soluble HSA when the solid composition is reconstituted in an aqueous medium, wherein less than 4.8% of soluble HSA contained in the solid composition is HSA dimer. In specific embodiments, less than 4.5%, less than 4%, less than 3.5%, less than 3%, less than 2.5%, less than 2%, less than 1.5%, less than 1%, or less than 0.5% of soluble HSA contained in the solid composition is HSA dimer. In specific embodiments, about 0.5% to about 1%, about 1% to about 1.5%, about 1.5% to about 2%, about 2% to about 2.5%, about 2.5% to about 3%, about 3% to about 3.5%, about 3.5% to about 3.6%, about 3.5% to about 4%, about 4% to about 4.5%, or about 4.5% to about 4.7% of soluble HSA contained in the solid composition is HSA dimer.

In specific embodiments, less than 4.6% of soluble HSA contained in the solid composition is HSA dimer, as determined using SEC by a 220 nm wavelength UV detector after at least a portion of the solid composition is reconstituted. In specific embodiments, less than 4.5%, less than 4%, less than 3.5%, less than 3%, less than 2.5%, less than 2%, less than 1.5%, less than 1%, or less than 0.5% of soluble HSA contained in the solid composition is HSA dimer, as determined using SEC by a 220 nm wavelength UV detector after at least a portion of the solid composition is reconstituted. In specific embodiments, about 0.5% to about 1%, about 1% to about 1.5%, about 1.5% to about 2%, about 2% to about 2.5%, about 2.5% to about 3%, about 3% to about 3.5%, about 3.5% to about 3.6%, about 3.5% to about 4%, or about 4% to about 4.5% of soluble HSA contained in the solid composition is HSA dimer, as determined using SEC by a 220 nm wavelength UV detector after at least a portion of the solid composition is reconstituted.

In specific embodiments, less than 3.7% of soluble HSA contained in the solid composition is HSA dimer, as determined using SEC by a 280 nm wavelength UV detector after at least a portion of the solid composition is reconstituted. In specific embodiments, less than 3.5%, less than 3%, less than 2.5%, less than 2%, less than 1.5%, less than 1%, or less than 0.5% of soluble HSA contained in the solid composition is HSA dimer, as determined using SEC by a 280 nm wavelength UV detector after at least a portion of the solid composition is reconstituted. In specific embodiments, about 0.5% to about 1%, about 1% to about 1.5%, about 1.5% to about 2%, about 2% to about 2.5%, about 2.5% to about 3%, about 3% to about 3.5%, or about 3.5% to about 3.6% of soluble HSA contained in the solid composition is HSA dimer, as determined using SEC by a 280 nm wavelength UV detector after at least a portion of the solid composition is reconstituted.

In specific embodiments, less than 4.8% of soluble HSA contained in the solid composition is HSA dimer, as determined using SEC by a fluorescence detector with an excitation wavelength of 280 nm and an emission wavelength of 350 nm after at least a portion of the solid composition is reconstituted. In specific embodiments, less than 4.5%, less than 4%, less than 3.5%, less than 3%, less than 2.5%, less than 2%, less than 1.5%, less than 1%, or less than 0.5% of soluble HSA contained in the solid composition is HSA dimer, as determined using SEC by a fluorescence detector with an excitation wavelength of 280 nm and an emission wavelength of 350 nm after at least a portion of the solid composition is reconstituted. In specific embodiments, about 0.5% to about 1%, about 1% to about 1.5%, about 1.5% to about 2%, about 2% to about 2.5%, about 2.5% to about 3%, about 3% to about 3.5%, about 3.5% to about 3.6%, about 3.5% to about 4%, about 4% to about 4.5%, or about 4.5% to about 4.7% of soluble HSA contained in the solid composition is HSA dimer, as determined using SEC by a fluorescence detector with an excitation wavelength of 280 nm and an emission wavelength of 350 nm after at least a portion of the solid composition is reconstituted.

The present invention also provides a solid composition comprising BoNT/A and HSA, wherein the HSA is formulated into the solid composition from a source material. As shown in Example 1, a BoNT/A composition dried without freezing not only has a smaller increase in the overall percentage of HSA aggregates over the HSA source material, but also has a smaller increase in the percentages of each of HSA polymer, oligomer, and dimer over the HSA source material, as compared with a BoNT/A composition dried using a process that involves freezing. In certain embodiments, the source material is a commercially available HSA product. In some embodiments, the source material is in a liquid form (e.g., in the form of a 25% solution). In other embodiments, the source material is in a solid form.

The percentage of HSA aggregates, polymer, oligomer, or dimer in soluble HSA contained in a solid composition described herein and the percentage of HSA aggregates, polymer, oligomer, or dimer in soluble HSA contained in the corresponding source material are preferably determined using the same method. When the source material of HSA is in a solid form, in certain embodiments the percentage of HSA aggregates, polymer, oligomer, or dimer in soluble HSA contained in the source material is determined after at least a portion of the source material is reconstituted, and reconstitution may be performed as described above.

In one aspect, provided herein is a solid composition comprising BoNT/A and HSA, wherein the HSA is formulated into the solid composition from a source material and comprises HSA monomer and HSA aggregates, wherein at least a portion of the HSA is in the form of soluble HSA when the solid composition is reconstituted in an aqueous medium, and wherein the percentage of HSA aggregates in soluble HSA contained in the solid composition is no more than 6.8-fold of the percentage of HSA aggregates in soluble HSA contained in the source material. In specific embodiments, the percentage of HSA aggregates in soluble HSA contained in the solid composition is no more than 6.5-fold, no more than 6-fold, no more than 5.5-fold, no more than 5-fold, no more than 4.5-fold, no more than 4-fold, no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA aggregates in soluble HSA contained in the source material. In specific embodiments, the percentage of HSA aggregates in soluble HSA contained in the solid composition is equal to or less than the percentage of HSA aggregates in soluble HSA contained in the source material. In specific embodiments, the percentage of HSA aggregates in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 3-fold, no more than 3- to 3.5-fold, no more than 3.5- to 4-fold, no more than 4- to 4.5-fold, no more than 4.5- to 5-fold, no more than 5- to 5.5-fold, no more than 5.5- to 6-fold, no more than 6- to 6.5-fold, or no more than 6.5- to 6.8-fold of the percentage of HSA aggregates in soluble HSA contained in the source material.

In specific embodiments, the percentage of HSA aggregates in soluble HSA contained in the solid composition is no more than 4.9-fold of the percentage of HSA aggregates in soluble HSA contained in the source material, as determined using SEC by a 220 nm wavelength UV detector after at least a portion of the solid composition is reconstituted. In specific embodiments, the percentage of HSA aggregates in soluble HSA contained in the solid composition is no more than 4.5-fold, no more than 4-fold, no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA aggregates in soluble HSA contained in the source material, as determined using SEC by a 220 nm wavelength UV detector after at least a portion of the solid composition is reconstituted. In specific embodiments, the percentage of HSA aggregates in soluble HSA contained in the solid composition is equal to or less than the percentage of HSA aggregates in soluble HSA contained in the source material, as determined using SEC by a 220 nm wavelength UV detector after at least a portion of the solid composition is reconstituted. In specific embodiments, the percentage of HSA aggregates in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 3-fold, no more than 3- to 3.5-fold, no more than 3.5- to 4-fold, no more than 4- to 4.5-fold, or no more than 4.5- to 4.9-fold of the percentage of HSA aggregates in soluble HSA contained in the source material, as determined using SEC by a 220 nm wavelength UV detector after at least a portion of the solid composition is reconstituted.

In specific embodiments, the percentage of HSA aggregates in soluble HSA contained in the solid composition is no more than 6.8-fold of the percentage of HSA aggregates in soluble HSA contained in the source material, as determined using SEC by a 280 nm wavelength UV detector after at least a portion of the solid composition is reconstituted. In specific embodiments, the percentage of HSA aggregates in soluble HSA contained in the solid composition is no more than 6.5-fold, no more than 6-fold, no more than 5.5-fold, no more than 5-fold, no more than 4.5-fold, no more than 4-fold, no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA aggregates in soluble HSA contained in the source material, as determined using SEC by a 280 nm wavelength UV detector after at least a portion of the solid composition is reconstituted. In specific embodiments, the percentage of HSA aggregates in soluble HSA contained in the solid composition is equal to or less than the percentage of HSA aggregates in soluble HSA contained in the source material, as determined using SEC by a 280 nm wavelength UV detector after at least a portion of the solid composition is reconstituted. In specific embodiments, the percentage of HSA aggregates in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, no more than 3- to 3.5-fold, no more than 3.5- to 4-fold, no more than 4- to 4.5-fold, no more than 4.5- to 5-fold, no more than 5- to 5.5-fold, no more than 5.5- to 6-fold, no more than 6- to 6.5-fold, or no more than 6.5- to 6.8-fold of the percentage of HSA aggregates in soluble HSA contained in the source material, as determined using SEC by a 280 nm wavelength UV detector after at least a portion of the solid composition is reconstituted.

In specific embodiments, the percentage of HSA aggregates in soluble HSA contained in the solid composition is no more than 1.9-fold of the percentage of HSA aggregates in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA aggregates in soluble HSA contained in the solid composition is no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA aggregates in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA aggregates in soluble HSA contained in the solid composition is equal to or less than the percentage of HSA aggregates in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA aggregates in soluble HSA contained in the solid composition is no more than 1.1- to 1.3-fold, no more than 1.3- to 1.5-fold, no more than 1.5- to 1.7-fold, or no more than 1.7- to 1.9-fold of the percentage of HSA aggregates in soluble HSA contained in the source material, as determined using SEC by a fluorescence detector with an excitation wavelength of 280 nm and an emission wavelength of 350 nm after at least a portion of the solid composition is reconstituted.

In another aspect, provided herein is a solid composition comprising BoNT/A and HSA, wherein the HSA is formulated into the solid composition from a source material, wherein at least a portion of the HSA is in the form of soluble HSA when the solid composition is reconstituted in an aqueous medium, and wherein the percentage of HSA polymer in soluble HSA contained in the solid composition is no more than 6.4-fold of the percentage of HSA polymer in soluble HSA contained in the source material. In specific embodiments, the percentage of HSA polymer in soluble HSA contained in the solid composition is no more than 6-fold, no more than 5.5-fold, no more than 5-fold, no more than 4.5-fold, no more than 4-fold, no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA polymer in soluble HSA contained in the source material. In specific embodiments, the percentage of HSA polymer in soluble HSA contained in the solid composition is equal to or less than the percentage of HSA polymer in soluble HSA contained in the source material. In specific embodiments, the percentage of HSA polymer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, no more than 3- to 3.5-fold, no more than 3.5- to 4-fold, no more than 4- to 4.5-fold, no more than 4.5- to 5-fold, no more than 5- to 5.5-fold, no more than 5.5- to 6-fold, or no more than 6- to 6.4-fold of the percentage of HSA polymer in soluble HSA contained in the source material.

In specific embodiments, the percentage of HSA polymer in soluble HSA contained in the solid composition is no more than 5.2-fold of the percentage of HSA polymer in soluble HSA contained in the source material, as determined using SEC by a 220 nm wavelength UV detector after at least a portion of the solid composition is reconstituted. In specific embodiments, the percentage of HSA polymer in soluble HSA contained in the solid composition is no more than 5-fold, no more than 4.5-fold, no more than 4-fold, no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA polymer in soluble HSA contained in the source material, as determined using SEC by a 220 nm wavelength UV detector after at least a portion of the solid composition is reconstituted. In specific embodiments, the percentage of HSA polymer in soluble HSA contained in the solid composition is equal to or less than the percentage of HSA polymer in soluble HSA contained in the source material, as determined using SEC by a 220 nm wavelength UV detector after at least a portion of the solid composition is reconstituted. In specific embodiments, the percentage of HSA polymer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, no more than 3- to 3.5-fold, no more than 3.5- to 4-fold, no more than 4- to 4.5-fold, no more than 4.5- to 5-fold, or no more than 5- to 5.2-fold of the percentage of HSA polymer in soluble HSA contained in the source material, as determined using SEC by a 220 nm wavelength UV detector after at least a portion of the solid composition is reconstituted.

In specific embodiments, the percentage of HSA polymer in soluble HSA contained in the solid composition is no more than 6.4-fold of the percentage of HSA polymer in soluble HSA contained in the source material, as determined using SEC by a 280 nm wavelength UV detector after at least a portion of the solid composition is reconstituted. In specific embodiments, the percentage of HSA polymer in soluble HSA contained in the solid composition is no more than 6-fold, no more than 5.5-fold, no more than 5-fold, no more than 4.5-fold, no more than 4-fold, no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA polymer in soluble HSA contained in the source material, as determined using SEC by a 280 nm wavelength UV detector after at least a portion of the solid composition is reconstituted. In specific embodiments, the percentage of HSA polymer in soluble HSA contained in the solid composition is equal to or less than the percentage of HSA polymer in soluble HSA contained in the source material, as determined using SEC by a 280 nm wavelength UV detector after at least a portion of the solid composition is reconstituted. In specific embodiments, the percentage of HSA polymer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, no more than 3- to 3.5-fold, no more than 3.5- to 4-fold, no more than 4- to 4.5-fold, no more than 4.5- to 5-fold, no more than 5- to 5.5-fold, no more than 5.5- to 6-fold, or no more than 6- to 6.4-fold of the percentage of HSA polymer in soluble HSA contained in the source material, as determined using SEC by a 280 nm wavelength UV detector after at least a portion of the solid composition is reconstituted.

In specific embodiments, the percentage of HSA polymer in soluble HSA contained in the solid composition is no more than 1.6-fold of the percentage of HSA polymer in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA polymer in soluble HSA contained in the solid composition is no more than 1.5-fold, no more than 1.4-fold, no more than 1.3-fold, no more than 1.2-fold, or no more than 1.1-fold of the percentage of HSA polymer in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA polymer in soluble HSA contained in the solid composition is equal to or less than the percentage of HSA polymer in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA polymer in soluble HSA contained in the solid composition is no more than 1.1- to 1.3-fold, no more than 1.3- to 1.5-fold, or no more than 1.5- to 1.6-fold of the percentage of HSA polymer in soluble HSA contained in the source material, as determined using SEC by a fluorescence detector with an excitation wavelength of 280 nm and an emission wavelength of 350 nm after at least a portion of the solid composition is reconstituted.

In another aspect, provided herein is a solid composition comprising BoNT/A and HSA, wherein the HSA is formulated into the solid composition from a source material, wherein at least a portion of the HSA is in the form of soluble HSA when the solid composition is reconstituted in an aqueous medium, and wherein the percentage of HSA oligomer in soluble HSA contained in the solid composition is no more than 3.9-fold of the percentage of HSA oligomer in soluble HSA contained in the source material. In specific embodiments, the percentage of HSA oligomer in soluble HSA contained in the solid composition is no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA oligomer in soluble HSA contained in the source material. In specific embodiments, the percentage of HSA oligomer in soluble HSA contained in the solid composition is equal to or less than the percentage of HSA oligomer in soluble HSA contained in the source material. In specific embodiments, the percentage of HSA oligomer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, no more than 3- to 3.5-fold, or no more than 3.5- to 3.9-fold of the percentage of HSA oligomer in soluble HSA contained in the source material.

In specific embodiments, the percentage of HSA oligomer in soluble HSA contained in the solid composition is no more than 3.9-fold of the percentage of HSA oligomer in soluble HSA contained in the source material, as determined using SEC by a 220 nm wavelength UV detector after at least a portion of the solid composition is reconstituted. In specific embodiments, the percentage of HSA oligomer in soluble HSA contained in the solid composition is no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA oligomer in soluble HSA contained in the source material, as determined using SEC by a 220 nm wavelength UV detector after at least a portion of the solid composition is reconstituted. In specific embodiments, the percentage of HSA oligomer in soluble HSA contained in the solid composition is equal to or less than the percentage of HSA oligomer in soluble HSA contained in the source material, as determined using SEC by a 220 nm wavelength UV detector after at least a portion of the solid composition is reconstituted. In specific embodiments, the percentage of HSA oligomer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, no more than 3- to 3.5-fold, or no more than 3.5- to 3.9-fold of the percentage of HSA oligomer in soluble HSA contained in the source material, as determined using SEC by a 220 nm wavelength UV detector after at least a portion of the solid composition is reconstituted.

In specific embodiments, the percentage of HSA oligomer in soluble HSA contained in the solid composition is no more than 3.9-fold of the percentage of HSA oligomer in soluble HSA contained in the source material, as determined using SEC by a 280 nm wavelength UV detector after at least a portion of the solid composition is reconstituted. In specific embodiments, the percentage of HSA oligomer in soluble HSA contained in the solid composition is no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA oligomer in soluble HSA contained in the source material, as determined using SEC by a 280 nm wavelength UV detector after at least a portion of the solid composition is reconstituted. In specific embodiments, the percentage of HSA oligomer in soluble HSA contained in the solid composition is equal to or less than the percentage of HSA oligomer in soluble HSA contained in the source material, as determined using SEC by a 280 nm wavelength UV detector after at least a portion of the solid composition is reconstituted. In specific embodiments, the percentage of HSA oligomer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, no more than 3- to 3.5-fold, or no more than 3.5- to 3.9-fold of the percentage of HSA oligomer in soluble HSA contained in the source material, as determined using SEC by a 280 nm wavelength UV detector after at least a portion of the solid composition is reconstituted.

In specific embodiments, the percentage of HSA oligomer in soluble HSA contained in the solid composition is no more than 3.9-fold of the percentage of HSA oligomer in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA oligomer in soluble HSA contained in the solid composition is no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA oligomer in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA oligomer in soluble HSA contained in the solid composition is equal to or less than the percentage of HSA oligomer in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA oligomer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, no more than 3- to 3.5-fold, or no more than 3.5- to 3.9-fold of the percentage of HSA oligomer in soluble HSA contained in the source material, as determined using SEC by a fluorescence detector with an excitation wavelength of 280 nm and an emission wavelength of 350 nm after at least a portion of the solid composition is reconstituted.

In another aspect, provided herein is a solid composition comprising BoNT/A and HSA, wherein the HSA is formulated into the solid composition from a source material, wherein at least a portion of the HSA is in the form of soluble HSA when the solid composition is reconstituted in an aqueous medium, and wherein the percentage of HSA dimer in soluble HSA contained in the solid composition is no more than 4.6-fold of the percentage of HSA dimer in soluble HSA contained in the source material. In specific embodiments, the percentage of HSA dimer in soluble HSA contained in the solid composition is no more than 4.5-fold, no more than 4-fold, no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA dimer in soluble HSA contained in the source material. In specific embodiments, the percentage of HSA dimer in soluble HSA contained in the solid composition is equal to or less than the percentage of HSA dimer in soluble HSA contained in the source material. In specific embodiments, the percentage of HSA dimer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, no more than 3- to 3.5-fold, no more than 3.5- to 4-fold, no more than 4- to 4.5-fold, or no more than 4.5- to 4.6-fold of the percentage of HSA dimer in soluble HSA contained in the source material.

In specific embodiments, the percentage of HSA dimer in soluble HSA contained in the solid composition is no more than 3.5-fold of the percentage of HSA dimer in soluble HSA contained in the source material, as determined using SEC by a 220 nm wavelength UV detector after at least a portion of the solid composition is reconstituted. In specific embodiments, the percentage of HSA dimer in soluble HSA contained in the solid composition is no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA dimer in soluble HSA contained in the source material, as determined using SEC by a 220 nm wavelength UV detector after at least a portion of the solid composition is reconstituted. In specific embodiments, the percentage of HSA dimer in soluble HSA contained in the solid composition is equal to or less than the percentage of HSA dimer in soluble HSA contained in the source material, as determined using SEC by a 220 nm wavelength UV detector after at least a portion of the solid composition is reconstituted. In specific embodiments, the percentage of HSA dimer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, or no more than 3- to 3.5-fold of the percentage of HSA dimer in soluble HSA contained in the source material, as determined using SEC by a 220 nm wavelength UV detector after at least a portion of the solid composition is reconstituted.

In specific embodiments, the percentage of HSA dimer in soluble HSA contained in the solid composition is no more than 4.6-fold of the percentage of HSA dimer in soluble HSA contained in the source material, as determined using SEC by a 280 nm wavelength UV detector after at least a portion of the solid composition is reconstituted. In specific embodiments, the percentage of HSA dimer in soluble HSA contained in the solid composition is no more than 4.5-fold, no more than 4-fold, no more than 3.5-fold, no more than 3-fold, no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA dimer in soluble HSA contained in the source material, as determined using SEC by a 280 nm wavelength UV detector after at least a portion of the solid composition is reconstituted. In specific embodiments, the percentage of HSA dimer in soluble HSA contained in the solid composition is equal to or less than the percentage of HSA dimer in soluble HSA contained in the source material, as determined using SEC by a 280 nm wavelength UV detector after at least a portion of the solid composition is reconstituted. In specific embodiments, the percentage of HSA dimer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, no more than 2.5- to 2.6-fold, no more than 2.5- to 3-fold, no more than 3- to 3.5-fold, no more than 3.5- to 4-fold, no more than 4- to 4.5-fold, or no more than 4.5- to 4.6-fold of the percentage of HSA dimer in soluble HSA contained in the source material, as determined using SEC by a 280 nm wavelength UV detector after at least a portion of the solid composition is reconstituted.

In specific embodiments, the percentage of HSA dimer in soluble HSA contained in the solid composition is no more than 2.6-fold of the percentage of HSA dimer in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA dimer in soluble HSA contained in the solid composition is no more than 2.5-fold, no more than 2-fold, no more than 1.9-fold, no more than 1.8-fold, no more than 1.7-fold, no more than 1.6-fold, no more than 1.5-fold, no more than 1.4-fold, no more than 1.3-fold, no more than 1.2-fold, or no more than 1.1-fold of the percentage of HSA dimer in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA dimer in soluble HSA contained in the solid composition is equal to or less than the percentage of HSA dimer in soluble HSA contained in the source material, as determined using SEC by a fluorescence detector with an excitation wavelength of 280 nm and an emission wavelength of 350 nm after at least a portion of the solid composition is reconstituted. In specific embodiments, the percentage of HSA dimer in soluble HSA contained in the solid composition is no more than 1.1- to 1.3-fold, no more than 1.3- to 1.5-fold, no more than 1.5- to 1.6-fold, no more than 1.5- to 1.7-fold, no more than 1.7- to 1.9-fold, no more than 1.1- to 1.5-fold, no more than 1.5- to 2-fold, no more than 2- to 2.5-fold, or no more than 2.5- to 2.6-fold of the percentage of HSA dimer in soluble HSA contained in the source material, as determined using SEC by a fluorescence detector with an excitation wavelength of 280 nm and an emission wavelength of 350 nm after at least a portion of the solid composition is reconstituted.

In various embodiments and aspects, provided herein is a solid composition comprising BoNT/A and HSA, wherein the solid composition has one, two, three, four, five, or more of the properties described above.

In various embodiments and aspects, provided herein is a solid composition comprising BoNT/A and HSA, wherein the solid composition is produced by a method described in Section 5.3.

In various embodiments and aspects, a solid composition described herein is animal product free. In various embodiments and aspects, a solid composition described herein is dried from a solution comprising BoNT/A and HSA, wherein the solution is animal product free. In various embodiments and aspects, a solid composition described herein can be reconstituted, for example, with water or saline, to a solution comprising BoNT/A and HSA, wherein the reconstituted solution is animal product free. In various embodiments and aspects, a solid composition described herein does not contain a protease inhibitor. In certain embodiments, a solid composition described herein does not contain benzamidine hydrocholoride. In various embodiments and aspects, a solution described herein comprising BoNT/A and HSA does not contain a protease inhibitor. In certain embodiments, a solution described herein comprising BoNT/A and HSA does not contain benzamidine hydrocholoride. In various embodiments and aspects, both the solid composition and the solution comprising BoNT/A and HSA do not contain a protease inhibitor. In certain embodiments, both the solid composition and the solution comprising BoNT/A and HSA do not contain benzamidine hydrochloride.

In various embodiments and aspects, the HSA described herein is recombinant HSA. In a specific embodiment, the recombinant HSA is animal product free. In a specific embodiment, the recombinant HSA is not produced from an animal. In a specific embodiment, the recombinant HSA is produced from a microorganism, such as bacteria. In a specific embodiment, the recombinant HSA is produced from a plant-based expression system. In various embodiments and aspects, the HSA described herein is human plasma-derived HSA. In a specific embodiment, the HSA contained in a solid composition described herein is about 0.5 mg per 100 units of BoNT/A.

In various embodiments and aspects, a solid composition described herein further comprises one or more pharmaceutically acceptable carriers. In various embodiments and aspects, a solution described herein comprising BoNT/A and HSA further comprises one or more pharmaceutically acceptable carriers. In specific embodiments, the one or more pharmaceutically acceptable carriers comprise sodium chloride (e.g., about 0.9 mg of sodium chloride per 100 units of BoNT/A). In specific embodiments, the solid composition described herein comprises about 0.9 mg of sodium chloride per 100 units of BoNT/A.

In various embodiments and aspects, a solid composition described herein is dried from a solution comprising BoNT/A and HSA in a manner such that the solution does not undergo freezing during drying.

In various embodiments and aspects, whether a solution described herein (or a reference solution described herein, as the case may be) undergoes freezing during drying is determined as described in Section 5.3.

In various aspects and embodiments, a solid composition described herein comprises about 50 units, about 100 units, or about 200 units of BoNT/A.

In various embodiments and aspects, a solid composition described herein has a potency of about 2.4×10⁷ units/mg to about 6.0×10⁷ units/mg.

The present invention provides a solid composition comprising BoNT/A and HSA that is dried without undergoing freezing. Such a solid composition has beneficial properties such as consistent visual product quality and a much slower potency decline during storage, e.g., at room temperature or higher, as compared to a reference solid composition stored under the same condition. A reference solid composition is dried from the same liquid composition as the studied solid composition, whereas they only differ in the drying process: the reference solid composition undergoes freezing conditions during the drying process. In other words, a reference solid composition has a higher percentage of HSA aggregates in HSA, a higher percentage of HSA aggregates, polymer, oligomer, or dimer in soluble HSA, and/or a higher percentage of insoluble HSA aggregates in HSA than the studied solid composition but is otherwise substantially identical to the studied solid composition. The term “substantially identical” is used in this context or the like to describe that the reference solid composition is identical or sufficiently similar to the solid composition (for example, in terms of their ingredients and the respective concentrations of the ingredients), such that the reference solid composition can serve the purpose of being an appropriate control for comparison. Potency of a solid composition that is dried without freezing reaches a steady state after storage for a period of time, and the steady-state potency of the solid composition is greater than the steady-state potency of a reference solid composition stored under the same condition. In certain embodiments, both the solid composition and the reference solid composition are stored at about 5° C., at about 25° C. (and optionally at about 60% relative humidity), or at about 40° C. (and optionally at about 75% relative humidity).

In various embodiments and aspects, potency of a solid composition described herein reaches a steady state after storage for a period of time, and wherein the steady-state potency of the solid composition is greater (e.g., at least 1-fold greater, at least 1.5-fold greater, at least 2-fold greater, at least 2.5-fold greater, at least 3-fold greater, at least 3.5-fold greater, at least 4-fold greater, at least 4.5-fold greater, or at least 5-fold greater) than the steady-state potency of a reference solid composition comprising BoNT/A and HSA under the same storage condition. In various embodiments and aspects, potency of a solid composition described herein reaches a steady state after storage for a period of time, and wherein the potency loss of the solid composition at the steady state relative to the beginning of the storage is less than (e.g., less than 10% of, less than 15% of, less than 20% of, less than 25% of, less than 30% of, less than 35% of, less than 40% of, less than 45% of less, less than 50% of, less than 55% of, less than 60% of, less than 65% of, less than 70% of, less than 75% of, less than 80% of, less than 85% of, less than 90% of, or less than 95% of) the potency loss of a reference solid composition.

Whether the potency of a solid composition has reached a steady state can be determined by a person of ordinary skill in the art. For example, the potency of a solid composition can be considered as having reached a steady state if potency of the solid composition does not decline for more than 20% after 3 months of storage.

In various embodiments and aspects, a solid composition described herein is a powdered pharmaceutical composition.

In another aspect, provided herein is a liquid composition comprising BoNT/A and HSA, which is derived from a solid composition described herein, for example, a dissolved or reconstituted form of a solid composition described herein.

In another aspect, provided herein is a method of treating a patient (preferably, a human patient) in need thereof, comprising administering a solid composition described herein.

### 5.4.1. Characterization of BoNT/A Compositions

In one embodiment, BoNT/A compositions described herein have a potency of at least about 1.5×10⁷ units/mg, e.g., about 1.5×10⁷ to about 6.0×10⁷ units/mg, about 2.0×10⁷ to about 6.0×10⁷ units/mg, about 2.4×10⁷ to about 6.0×10⁷ units/mg, about 2.4×10⁷ to about 5.9×10⁷ units/mg, about 2.4×10⁷ to about 5.8×10⁷ units/mg, about 2.4×10⁷ to about 5.7×10⁷ units/mg, about 2.4×10⁷ to about 5.6×10⁷ units/mg, about 2.4×10⁷ to about 5.5×10⁷ units/mg, about 2.4×10⁷ to about 5.4×10⁷ units/mg, about 2.5×10⁷ to about 6.0×10⁷ units/mg, about 2.6×10⁷ to about 6.0×10⁷ units/mg, about 2.7×10⁷ to about 6.0×10⁷ units/mg, about 2.8×10⁷ to about 6.0×10⁷ units/mg, about 2.9×10⁷ to about 6.0×10⁷ units/mg, about 3.0×10⁷ to about 6.0×10⁷ units/mg, or any numbers between such ranges.

In one embodiment, BoNT/A compositions described herein have a potency of about 2.4×10⁷ units/mg to about 5.4×10⁷ units/mg. In preferred embodiments, the term “unit” used herein refers to the LD₅₀ dose.

In various embodiments and aspects, a solid composition described herein is a dried product of a solution comprising BoNT/A and HSA and has a higher potency than a reference solid composition, wherein the reference solid composition is produced from a reference solution comprising BoNT/A and HSA by a drying process performed in a manner such that the reference solution undergoes freezing during drying, and wherein the reference solution is substantially identical to the solution. The term “substantially identical” can be understood as described above. Further description regarding a reference solid composition is provided above in Section 5.3. In certain embodiments, the solid composition described herein has a potency that is at least 10%, at least 20%, at least 30%, at least 40%, at least 50%, at least 60%, at least 70%, at least 80%, at least 90%, at least 100%, at least 1.5-fold, or at least 2-fold higher than the potency of the reference solid composition. In certain embodiments, the solid composition described herein has a potency that is at least 0.1×10⁷ units/mg, at least 0.2×10⁷ units/mg, at least 0.3×10⁷ units/mg, at least 0.4×10⁷ units/mg, at least 0.5×10⁷ units/mg, at least 0.6×10⁷ units/mg, at least 0.7×10⁷ units/mg, at least 0.8×10⁷ units/mg, at least 0.9×10⁷ units/mg, at least 1×10⁷ units/mg, at least 1.5×10⁷ units/mg, at least 2×10⁷ units/mg, at least 3×10⁷ units/mg, or at least 4×10⁷ units/mg higher than the potency of the reference solid composition.

In various embodiments and aspects, a solid composition described herein is a dried product of a solution comprising BoNT/A and HSA and has a slower potency decline than a reference solid composition, wherein the reference solid composition is produced from a reference solution comprising BoNT/A and HSA by a drying process performed in a manner such that the reference solution undergoes freezing during drying, and wherein the reference solution is substantially identical to the solution. The term “substantially identical” can be understood as described above. Further description regarding a reference solid composition is provided above in Section 5.3. In certain embodiments, the solid composition described herein has an at least 10%, at least 20%, at least 30%, at least 40%, at least 50%, at least 60%, at least 70%, at least 80%, at least 90%, or at least 95% slower potency decline relative to the reference solid composition.

The potency of the BoNT/A compositions described herein can be determined with methods known in the art, including but not limited to, e.g., Light-Chain Activity High-Performance Liquid Chromatography (LCA-HPLC) assay, mouse 50% lethal dose (MLD₅₀) assay, Mouse Digit Abduction Score (DAS) assay, SNAP-25 assay, cell-based potency assay (CBPA), etc.

The LCA-HPLC assay measures SNAP-25 cleavage specificity. Samples are reacted with a commercially available BoNT/A fluorescent substrate derived from the SNAP-25 sequence. The fluorescently-labeled cleavage products are separated and detected via a reverse-phase (RP)-HPLC method. Further description of LCA-HPLC assay can be found in publications Hunt et al. (2010) Toxins 2(8):2198-2212 and Rupp et al. (2020) Toxins 12(6):393, each of which is incorporated herein by reference in its entirety.

In one embodiment, the potency is determined using a mouse 50% lethal dose (MLD50) assay. The mouse 50% lethal dose (MLD50) assay has been described in, e.g., Schantz and Kautter (1978) Journal of the AOAC 61(1):96-99, Hunt and Kenneth (2009) Clinical Neuropharmacology 32(1):28-31, U.S. Pat. Nos. 7,160,699, and 9,725,705, each of which is incorporated herein by reference in its entirety. Mouse 50% lethal dose (MLD50) assay is a method for measuring the potency of a botulinum toxin by intraperitoneal injection of the botulinum toxin into female mice (about four weeks old) weighing 17-22 grams each at the start of the assay. Each mouse is held in a supine position with its head tilted down and is injected intraperitoneally into the lower right abdomen at an angle of about 30 degrees using a 25 to 27 gauge ⅜″ to ⅝″ needle with one of several serial dilutions of the botulinum toxin in saline. The death rates over the ensuing 72 hours for each dilution are recorded. The dilutions are prepared so that the most concentrated dilution produces a death rate of at least 80% of the mice injected, and the least concentration dilution produces a death rate of no greater than 20% of the mice injected. There must be a minimum of four dilutions that fall within the monotone decreasing range of the death rates. The monotone decreasing range commences with a death rate of no less than 80%. Within the four or more monotone decreasing rates, the two largest and the two smallest rates must be decreasing (i.e., not equivalent). The dilution at which 50% of the mice die within the three day post injection observation period is defined as a dilution which comprises one unit (1 U) of the botulinum toxin.

Mouse Digit Abduction Score (DAS) assay is an in vivo assessment of toxin-induced muscle paralysis following injection of BoNT/A toxin into the hind limb muscle of a rodent. The DAS assay can be used to assess the potency of BoNT/A compositions on muscle paralysis, as well as the duration of action. Detailed protocols of DAS assay have been disclosed in Aoki et al. (1999) Eur. J. Neurol. 6: s3-s10, Aoki (2001) Toxicon 39:1815-1820, Broide et al. (2013) Toxicon 71:18-24, and Rupp et al. (2020) Toxins 12 (6): 393, each of which is incorporated herein by reference in its entirety. For example, the DAS assay can be performed by injection of a BoNT/A composition described herein into the mouse gastrocnemius/soleus complex, followed by assessment of Digital Abduction Score using the method of Aoki (2001) Toxicon 39:1815-1820. In the DAS assay, mice are suspended briefly by the tail in order to elicit a characteristic startle response in which the mouse extends its hind limbs and abducts its hind digits. Following the BoNT/A composition injection, the varying degrees of digit abduction are scored on a five-point scale (0-normal to 4-maximal reduction in digit abduction and leg extension). Safety Ratio, the ratio between the amount of a toxin required for a 10% drop in a bodyweight (measured at peak effect within the first seven days after dosing in a mouse) and the amount of toxin required for a DAS score of 2, can also be determined to assess the therapeutic index of the BoNT/A composition described herein, as described in U.S. Pat. No. 9,920,310, which is incorporated by reference herein in its entirety. High Safety Ratio scores are therefore desired, and indicate a toxin that is able to effectively paralyze a target muscle with little undesired off-target effects.

SNAP-25 assay is an ELISA based method to measure SNAP-25 proteolytic activity of the botulinum toxin. The assay uses a truncated SNAP-25 protein (the 206 amino acid residue peptide) bound to polystyrene 96 well microtiter plates and a monoclonal antibody that recognizes the cleaved product (a 197 amino acid residue peptide) which is made by enzymatic hydrolysis between amino acids 197 and 198 of the SNAP-25 by reduced botulinum toxin type A. The monoclonal antibody bound to the cleaved product is then detected with a secondary antibody (goat anti-mouse IgG conjugated to horseradish peroxidase HRP), which produces a color change in the presence of a chromogenic substrate (TMB). Exemplary SNAP-25 methods are described in Ekong et al. (1997) Microbiology 143:3337-3347, and U.S. Pat. No. 7,160,699, each of which is incorporated herein by reference.

Cell-based potency assay (CBPA) has been described in, e.g., Fernández-Salas et al. (2012) PLOS ONE 7(11):e49516, Rupp et al. (2020) Toxins 12 (6): 393, WO 2010/105234, and WO 2009/114748, each of which is incorporated herein by reference. In one embodiment, the SNAP-25₁₉₇ SiMa H1 electrochemiluminescent (ECL) CBPA is used to determine the potency of the BoNT/A compositions described herein. The SNAP-25₁₉₇ SiMa H1 electrochemiluminescent (ECL) CBPA is an in vitro cell-based assay that measures the key steps of BoNT/A intoxication: receptor-mediated cell binding and internalization, translocation of the protease domain (light chain) into the cytosol, and proteolytic cleavage of SNAP-25, allowing direct comparison of BoNT/A product biological activity in vitro (Fernández-Salas et al. (2012) PLOS ONE 7 (11): e49516; Rupp et al. (2020) Toxins 12(6):393). In brief, human neuroblastoma SiMa H1 cells are plated onto poly-D-lysine (PDL) 96-well plates in serum-free media (SFM) with 25 μg/mL of GT_{1b} for three days and treated with toxin samples for 24 hours. After treatment, toxins are removed, cells are lysed and lysates are transferred to MSD High Bind plates coated with anti-SNAP-25₁₉₇ monoclonal antibody (mAb) 2E2A6. Plates are then washed and incubated with SULFO-TAG NHS-Ester labeled anti-SNAP-25 polyclonal antibody (pAb) for detection. Captured, BoNT/A toxin-cleaved SNAP-25 is then quantitated on a MSD plate reader.

In various embodiments and aspects, a solid composition described herein is a dried product of a solution comprising BoNT/A and HSA and has a higher visual product quality (e.g., has less or no flaked product) relative to a reference solid composition, wherein the reference solid composition is produced from a reference solution comprising BoNT/A and HSA by a drying process performed in a manner such that the reference solution undergoes freezing during drying, and wherein the reference solution is substantially identical to the solution. In various embodiments and aspects, a solid composition described herein is a dried product of a solution comprising BoNT/A and HSA and has a more consistent visual product quality (e.g., more consistently has less or no flaked product) relative to a reference solid composition. The term “substantially identical” can be understood as described above. Further description regarding a reference solid composition is provided above in Section 5.3.

In various embodiments and aspects, a solid composition described herein is a dried product of a solution comprising BoNT/A and HSA and has a lower immunogenicity than a reference solid composition, wherein the reference solid composition is produced from a reference solution comprising BoNT/A and HSA by a drying process performed in a manner such that the reference solution undergoes freezing during drying, and wherein the reference solution is substantially identical to the solution. The term “substantially identical” can be understood as described above. Further description regarding a reference solid composition is provided above in Section 5.3.

Immunogenicity of a BoNT/A composition can be measured by any method described herein or known in the art suitable for measuring the immunogenicity of a BoNT/A composition, such as, but is not limited to, the detection of neutralizing antibodies (e.g., anti-HSA antibodies and/or anti-BoNT/A antibodies).

### 6. ILLUSTRATIVE EMBODIMENTS

The present disclosure includes the following non-liming illustrative embodiments:
1. A solid composition comprising a 900 kDa Clostridium botulinum neurotoxin serotype A (BoNT/A) complex, human serum albumin (HSA), and sodium chloride (NaCl), wherein the HSA comprises HSA monomer and HSA aggregates, wherein at least a portion of the HSA is in the form of soluble HSA when the solid composition is reconstituted in an aqueous medium, and wherein less than 34% of soluble HSA contained in the solid composition are HSA aggregates.
2. The solid composition of embodiment 1, wherein the HSA aggregates comprise HSA polymer, HSA oligomer, and HSA dimer.
3. The solid composition of embodiment 1 or embodiment 2, wherein less than 30% of the soluble HSA contained in the solid composition is HSA polymer.
4. The solid composition of embodiment 1 or embodiment 2, wherein less than 0.8% of soluble HSA contained in the solid composition is HSA oligomer.
5. The solid composition of embodiment 1 or embodiment 2, wherein less than 4.8% of soluble HSA contained in the solid composition is HSA dimer.
6. The solid composition of any one of embodiments 1 to 5, wherein the percentage of the HSA aggregates, the HSA polymer, the HSA oligomer, and/or the HSA dimer in the soluble HSA contained in the solid composition is determined using size exclusion chromatography (SEC) after at least a portion of the solid composition is reconstituted with water.
7. The solid composition of any one of embodiments 1 to 6, wherein the solid composition is a powdered pharmaceutical composition.
8. The solid composition of any one of embodiments 1 to 7, wherein the percentage of the HSA aggregates, the HSA polymer, the HSA oligomer, and/or the HSA dimer in the soluble HSA contained in the solid composition is determined using size exclusion chromatography (SEC) by a 220 nm wavelength UV detector after at least a portion of the solid composition is reconstituted with water.
9. The solid composition of any one of embodiments 1 to 8, wherein the solid composition is animal product free.
10. The solid composition of any one of embodiments 1 to 9, wherein the HSA is recombinant HSA.
11. The solid composition of any one of embodiments 1 to 10, wherein the solid composition comprises about 0.5 mg of HSA per 100 units of BoNT/A.
12. The solid composition of any one of embodiments 1 to 11, wherein the solid composition comprises about 0.9 mg of NaCl per 100 units of BoNT/A.
13. The solid composition of any one of embodiments 1 to 12, wherein the BoNT/A is onabotulinumtoxinA.
14. The solid composition of any one of embodiments 1 to 13, where the BoNT/A is produced by a column chromatography purification process.
15. The solid composition of any one of embodiments 1 to 14, where the BoNT/A is produced by a column chromatography purification process, wherein the column chromatography purification process comprises hydrophobic interaction chromatography.
16. The solid composition of any one of embodiments 1 to 15, wherein the column chromatography purification process further comprises anion exchange chromatography.
17. The solid composition of any one of embodiments 1 to 16, wherein the column chromatography purification process further comprises cation exchange chromatography.
18. The solid composition of any one of embodiments 1 to 17, wherein the solid composition does not comprise a protease inhibitor.
19. The solid composition of any one of embodiments 1 to 18, wherein the solid composition does not comprise benzamidine hydrocholoride.
20. The solid composition of any one of embodiments 1 to 19, wherein the solid composition has a potency of about 1.5×10⁷ units/mg to about 6.0×10⁷ units/mg.
21. A method of producing a solid composition comprising a 900 kDa BoNT/A complex, HSA, and NaCl, said method comprising a step of drying a solution comprising BoNT/A, HSA, and NaCl to produce said solid composition, wherein the solution does not undergo freezing during the drying step.
22. The method of embodiment 21, wherein the step of drying is at least partially manually controlled.
23. The method of embodiment 21 or embodiment 22, wherein the step of drying is vacuum drying.
24. The method of embodiment 23, wherein the vacuum drying step comprises a stepwise evacuating step.
25. The method of any one of embodiments 21-24, wherein the method produces a solid composition according to any one of embodiments 1-20.
26. A solid composition produced by the method of any one of embodiments 21 to 25.
27. A method of treating a patient in need thereof, comprising administering the solid composition of any one of embodiments 1-20 and 26.
28. A method substantially as described herein.
29. A composition substantially as described herein.

### 7. EXAMPLE

Certain embodiments provided herein are illustrated by the following non-limiting examples, which describe different methods for obtaining BoNT/A and demonstrate that freezing during drying results in a significant increase in the amount of HSA aggregation in a solid BoNT/A composition, as compared to drying without freezing, and that solid BoNT/A compositions dried without undergoing freezing have a slower potency decline during storage.

### 7.1. Example 1-Chemical and Physical Stability of Human Serum Albumin During Manufacture of Botulinum Toxin Drug Product: Laboratory-Scale Investigation

### 7.1.1. Introduction

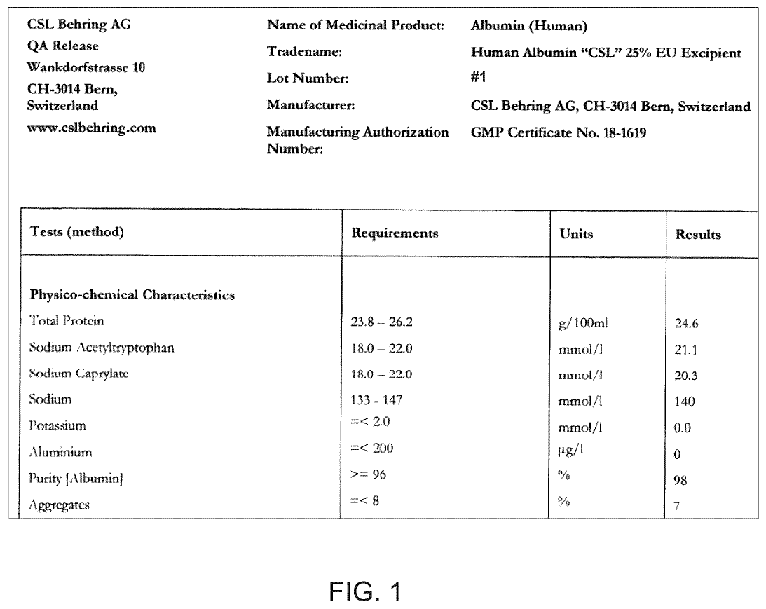

The majority of commercial neurotoxin drug products contain human serum albumin (HSA) as an excipient. Understanding HSA aggregation could be important in development and commercialization of neurotoxin products. HSA aggregates may be introduced to drug product (DP) from an incoming HSA that is used in DP manufacture. HSA monograph for European Pharmacopoeia specifies that the total area of peaks due to HSA polymers and aggregates should not be greater than 10% (http://www.uspbpep.com/ep60/human %20albumin %20 solution %200255e.pdf, EUROPEAN PHARMACOPOEIA 6.0, Human Albumin Solution). Manufacturers of pharmaceutical-grade HSA may also impose tighter restrictions on the aggregation level. For example, one of the HSA vendors, CSL Berring, specifies the requirement for aggregates as ≤8% (FIG. 1). This study investigated whether HSA aggregates might also be produced during DP manufacture. As with the manufacture of any sterile biopharmaceutical product, there are several steps in the manufacture of a BoNT/A DP.

In the instant study, the aggregation of HSA was measured in laboratory-produced BoNT/A DP lots and placebo lots that were manufactured using three sets of conditions using the same formulation, containing HSA and NaCl, with (for BoNT/A DP) and without (for placebo) the active ingredient, BoNT/A. The main differences in the manufacturing processes include drying process and hold of the bulk solution before the drying step. Commercial BoNT/A drug products are produced either with lyophilization (freeze-drying) or vacuum drying. Both manufacturing methods can utilize the same freeze-drying equipment, while the main difference is the freezing step used in the former. However, evaporating cooling during vacuum drying can also result in freezing.

Another potential difference in the manufacturing conditions is the time period during which a bulk drug product is stored before initiation of sterilizing filtration, and also from the filtration to completion of vial filling. While such hold times for laboratory manufacture, where tens to a few hundred vials are produced, can be minimized to less than an hour, pre-drying hold time in commercial production, when the number of vials produced counts to thousands and tens of thousands, would be much longer. In addition, a typical commercial process is validated for extended hold times to account for a possibility of unexpected delays, e.g., a malfunctioning of filling equipment. Accordingly, the impact of the pre-drying hold of the bulk drug product solution was also evaluated in this study. The formulation contains HSA and NaCl. Lots were produced with the active ingredient, BoNT/A (active DP lots) and without BoNT/A (placebo lots).

### 7.1.2. Materials and Methods

### 7.1.2.1. Materials

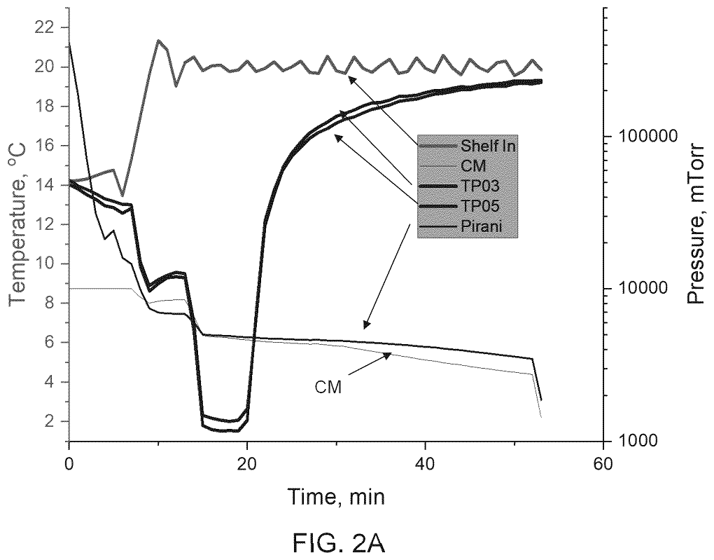

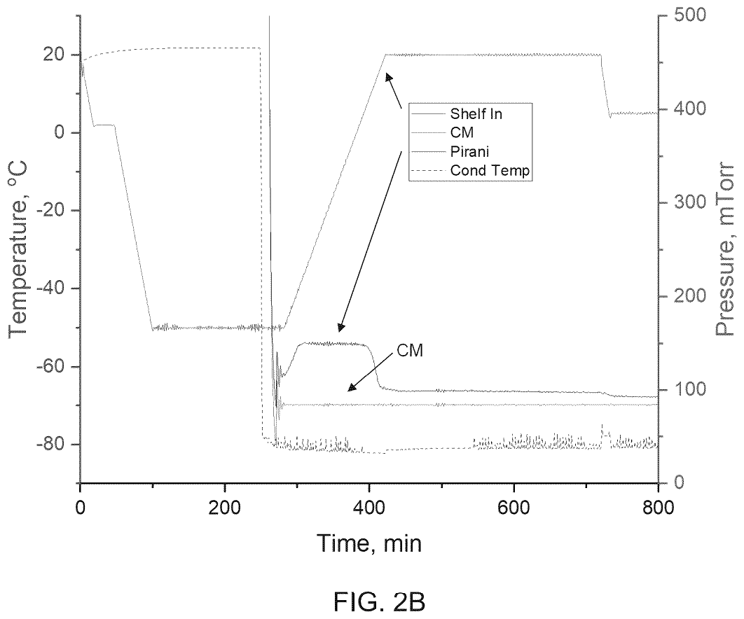

HSA was sourced from CSL Behring as a 25% solution. Sterile NaCl 0.9 wt % solution (USP, EP, BP, JP grade, Terumo BCT Ltd), NaCl granules (JT Baker, USP, EP, JP, BP grade), and MilliQ water were used for manufacturing. A concentrated solution of BoNT/A drug substance (DS) was provided by AbbVie. Vials (glass type 1, 10 ml, Gerresheimer) were washed and depyrogenated. Stoppers (20 mm Lyotec West S-87-J, 4432/50 GRY, ready-to-sterilize) were sterilized and dried. For lots with an active ingredient, solution of BoNT/A drug substance was added to the excipient solution (0.5 wt % HSA and 0.9 wt % NaCl) using a dilution factor of 23000, filtered using gamma-irradiated Millipak-20 0.2 μm sterilizing filter, aseptically filled into 10 ml glass type 1 vials at 0.1 ml/vial, partially stoppered, loaded into freeze-dryer LyoStar2, vacuum-dried or freeze-dried, stoppered under vacuum at the end of the drying process, and sealed with aluminum shell. The placebo lots were manufactured similarly, with the exception of the DS addition and a higher fill volume of 0.2 ml/vial. Product temperature during vacuum drying was measured with temperature probes (Ellab, Denmark) in real time; the temperature data were also recorded with the data loggers. Three sets of manufacturing conditions were used, with main differences depicted in Table 2 and more details explained in Section 7.1.2.2 below. Five drug product lots and three placebo lots were manufactured. FIGS. 2A and 2B show representative vacuum-dried and freeze-dried cycles.

TABLE 2
Main differences in the manufacturing conditions
of the laboratory drug product lots.

| | | | Number of |
|---|---|---|---|
| Manufacturing | Pre-/pos filtration | | lots produced, |
| process ID | hold of the bulk DP | Drying method | active/placebo |
| 1 | <15 min/<15 min | Vacuum-drying | 2/1 |
| 2 | <15 min/<15 min | Freeze-drying | 1/1 |
| 3 | 24 h/24 h | Freeze-drying | 2/1 |

### 7.1.2.2. Methods

### 7.1.2.2.1. Drying Procedures

The process-1 (i.e., vacuum drying) cycle is presented in Table 3. Evacuation of the lyophilizer causes the vials to cool down. To prevent freezing of the solutions in the vials, a specific stepwise procedure was adopted that allowed the lyophilizer chamber to be evacuated without causing solutions in the vials to freeze. Specifically, before the automated vacuum-drying cycle in Table 3 was initiated (i.e., before the “Vacuum ‘On’” step and after the “Pre-Freeze” step), the vacuum was controlled manually, as follows, to minimize the risk of the solutions in the vials freezing due to rapid evacuation of the lyophilizer sample chamber. The main reason for this stepwise manual control is the fact that the evacuation rate cannot be controlled on Lyostar2 during the cycle. Instead, the vacuum was turned on, with the evacuation rate uncontrolled, and was stopped at various intervals to allow the product temperature to go back up to a higher temperature (such as 20° C.) before the vacuum was turned on again. Two temperature thermocouples were inserted into 2 vials near the middle of the tray, to monitor product temperature. The manual cycle was turned on. The shelf temperature was set to 20° C. The condenser was turned on. Vacuum was then turned on and manually stopped at 20,000 mTorr. The product temperature, obtained from the thermocouple readings, was 16° C. The product temperature was allowed to go up to 20° C. and evacuation resumed until the Pirani pressure reading was 5,000 mTorr, at which point the vacuum was turned off. The product temperature was 2° C. Once again, the product temperature was allowed to rise to 18° C. At this point, the automated cycle presented in Table 3, below, was initiated. Once the vacuum-drying cycle was completed, the vials were fully stoppered, capped and stored in a freezer at −20° C.

TABLE 3
Process-1 cycle parameters.

| | | | Hold | |
|---|---|---|---|---|
| | Temperature, | Ramp Rate, | Time, | Pressure, |
| Step | ° C. | ° C./min | min. | mTorr |
| Pre-Freeze | 20 | 1 | 15 | Ambient |
| Freezing | NA | NA | NA | Ambient |
| Extra Freeze Time | NA | NA | NA | Ambient |
| Vacuum “On” | 20 | 0 | 15 | 84 |
| Primary Drying | 20 | 1 | 300 | 84 |
| Secondary Drying | NA | NA | NA | NA |
| Hold | 5 | NA | NA | 84 |

NA = Not Applicable

The process-2 (i.e., freeze drying) cycle is presented in Table 4. Specifically, a freezing cycle at −50° C., for 120 minutes, was included in this cycle. The process-2 cycle did not include a stepwise evacuation step. Once the cycle was completed, the vials were fully stoppered under vacuum and the lyophilizer was brought back to ambient conditions. The vials were capped and stored at −20° C. until testing.

TABLE 4
Process-2 cycle parameters.

| | | | Hold | |
|---|---|---|---|---|
| | Temperature, | Ramp Rate, | Time, | Pressure, |
| Step | ° C. | ° C./min | min. | mTorr |
| Pre-Freeze | 2 | 1 | 15 | Ambient |
| Freezing | −50 | 1 | 120 | Ambient |
| Extra Freeze Time | NA | NA | NA | Ambient |
| Vacuum “On” | −50 | 0 | 15 | 84 |
| Primary Drying | 20 | 1 | 300 | 84 |
| Secondary Drying | NA | NA | NA | NA |
| Hold | 5 | NA | NA | 84 |

NA = Not Applicable

The process-3 (i.e., freeze drying plus holds at room temperature) cycle is presented in Table 5. Specifically, a freezing cycle at −50° C., for 120 minutes, was included in this cycle. The process-3 cycle did not include a stepwise evacuation step. Once the cycle was completed, the vials were fully stoppered under vacuum and the lyophilizer was brought back to ambient conditions. The vials were capped and stored at −20° C. until testing. Before the drying cycle, the product solution was stored at room temperature for 24 hours before filtration and for another 24 hours after filtration.

TABLE 5
Process-3 cycle parameters.

| | | | Hold | |
|---|---|---|---|---|
| | Temperature, | Ramp Rate, | Time, | Pressure, |
| Step | ° C. | ° C./min | min. | mTorr |
| Pre-Freeze | 2 | 1 | 15 | Ambient |
| Freezing | −50 | 1 | 120 | Ambient |
| Extra Freeze Time | NA | NA | NA | Ambient |
| Vacuum “On” | −50 | 0 | 15 | 84 |
| Primary Drying | 20 | 1 | 300 | 84 |
| Secondary Drying | NA | NA | NA | NA |
| Hold | 5 | NA | NA | 84 |

NA = Not Applicable

### 7.1.2.2.2. Analysis of HSA Aggregation by SEC

Human serum albumin (HSA) aggregation was measured by size exclusion chromatography (SEC).

The dried samples were reconstituted by adding 0.25 ml water per vial. The 25% neat HSA solutions were diluted with water to 0.25 mg/mL prior to injection on HPLC. System suitability standards were injected 5 times prior to sample injections and subsequently every 10 injections for system suitability. The HPLC parameters are provided in Table 6. Samples were run and analyzed using Empower. Five active and three placebo lots were tested by SEC. Statistical analysis of the SEC data was performed using the JMP 10.0 statistical software.

TABLE 6
SEC Parameters.

| Parameter | Method |
|---|---|
| Injection Volume | 50 uL |
| Column Temperature | Room temperature |
| Column | Superdex 200 10/300 |
| Flow Rate | 0.5 mL/min |
| Mobile Phase | 50 mM Potassium Phosphate + |
| | 150 mM NaCl, pH 7.0 |
| Run Time | 60 minute |
| Sample Prep | Reconstitute vials to 0.25 mg/mL of water |
| Detection | (a) 280 nm |
| | (b) 220 nm |
| | (c) Fluorescence (Excitation |
| | 280 nm, Emission 350 nm) |

### 7.1.2.2.3. Karl Fischer Titration

Coulometric Karl Fischer titration was used to quantify residual water content in the dried product. KF titrator with oven and automatic sampler was utilized. Samples were analyzed in triplicate. Sealed vials were heated in the KF oven at 115° C. Nitrogen gas was used to purge the vials and carry any moisture in the headspace to the reaction vessel, containing the KF reagent. The reaction would proceed until the rate dropped below 20 μg/min. Empty vials, which went through the same freeze-drying or vacuum-drying cycles, were also tested, and the water content in the blank was subtracted to obtain water content in the dried material. Four representative lots were tested.

### 7.1.2.2.4. Scanning Electron Microscopy (SEM)

Three lots of placebo dried cake were examined using an FEI Quanta-450 (Thermo Fisher Scientific, Hillsboro, OR) field emission SEM with a backscattered electron detector. Imaging conditions were 10 kV and 10-mm working distance in low vacuum. The samples were uncoated. SEM imaging of the placebo dried cake morphology required breakage of the glass vial to isolate the dried cake/glass base portions. This was accomplished by scoring the vial near the heel using a diamond scribe and then breaking the glass using a sharp impact. Appropriate portions of each sample were then mounted on an SEM stub using carbon tape. Three placebo lots were tested by SEM.

### 7.1.2.2.5. Subvisible Particle Test

For subvisible particle test (also referred to as subvisible particulate test), three placebo lots, which were produced by processes 1, 2, and 3, respectively, were tested. Vials with dried placebo were reconstituted by adding 2 mL saline per vial and gently mixed. Vials from each lot were tested with a lab-built noninvasive subvisible particle imaging system as described in U.S. Pat. No. 10,132,736 B2, which is incorporated by reference herein in its entirety. During the particle measurement, a reconstituted solution in vial was partially illuminated with a laser beam focused into a thin light sheet. Video images were recorded with a sensitive digital video camera with a specially designed lens system to compensate the image distortion caused by the cylinder shape of the container. By using this system, subvisible particles can be counted noninvasively in syringes or vials. The in-lab-developed software can count the particle numbers, sizes and intensities in each image frame in real time. Absolute particle number and size can be calibrated using standard particle samples.

### 7.1.2.2.6. XRPD

The XRPD diffractograms were collected on a MiniFlex 600 X-ray diffractometer (Rigaku, Texas US) with Cu Kα radiation (λ=1.54 Å, 40 kV/15 mA). The instrument was calibrated with a silicon standard with a reference peak at 28.44° (2θ). Solid samples were prepared in a low background Si holder by applying gentle pressure to keep the sample surface flat and level with the reference surface of the sample holder. Each sample was analyzed from 3 to 45° (2θ) using a continuous scan of 1° (2θ) per minute with a step size of 0.01° (20). Data analysis was performed on the PDXL software.

### 7.1.2.2.7. Small-Angle X-Ray Scattering/Wide-Angle X-Ray Scattering (SAXS/WAXS)

The SAXS/WAXS tests of two solutions (0.9% NaCl/0.5% HSA and 0.9% NaCl in water) were performed on ID02 beamline at the European Synchrotron Radiation Facility (ESRF). The solutions were filled into 1 mm capillaries, and loaded into Linkam stage for variable temperatures runs. Two-dimensional SAXS and WAXS images were collected using two different detectors. The exposure time was adjusted to use the maximum dynamic of the detectors for every sample and was less than 1 second in the majority of cases. The two-dimensional images were normalized to an absolute intensity scale after performing the standard detector corrections and azimuthally integrated to obtain the corresponding one-dimensional X-ray diffraction curves. The SAXS and WAXS q-scales were calibrated with silver behenate and silicon powders, respectively. Combined SAXS/WAXS measurements were all normalized.

### 7.1.2.2.8. Changes in Apparent pH

To monitor changes in the apparent pH during freezing of the formulation, a low-temperature pH electrode, InLab®Cool (pH 1 to 11, temperature −30° C. to 80° C., Electrolyte PRISCOLYT-B®) (METTLER TOLEDO), was used. The pH electrode was calibrated at room temperature using NIST tracable buffer standards-pH 4.01, pH 7.00 and pH 10.00 (Aqua solutions). A glass beaker with 50 mL of placebo solution was transferred into a lyophilizer (Lyostar III, SP Scientific). Temperature probe and the low-temperature pH electrode were placed in the placebo solution beaker. The lyophilizer door was closed and cooling was started in manual mode with shelf temperature set at −55° C. The pH reading was recorded at each unit change in sample temperature. After the sample temperature reached-50° C., shelf set point was increased in increments of 10° C. until sample temperature reached within 2° C. of shelf temperature allowing the sample to thaw to 20° C. The pH was also measured during evaporation, using micro electrode InLab®Micro (pH 0 to 14, temperature 0° C. to 80° C., Electrolyte 3 M KCl) (METTLER TOLEDO), while sample weight was determined at regular intervals with the placebo evaporating under nitrogen overflow. The pH micro-electrode was calibrated at room temperature using the same NIST traceable buffer standards mentioned above. Approximately 7.3 g of placebo solution was placed in a glass vial and exposed to nitrogen overflow in a fume hood. The sample vial was removed from the hood intermittently to record pH and weight data.

### 7.1.3. Results

### 7.1.3.1. Characterization of the “Neat” HSA.

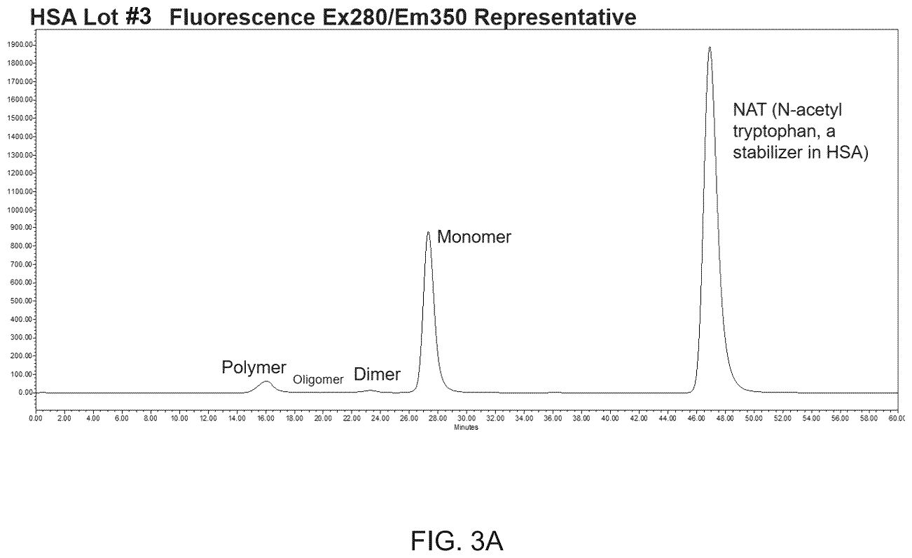

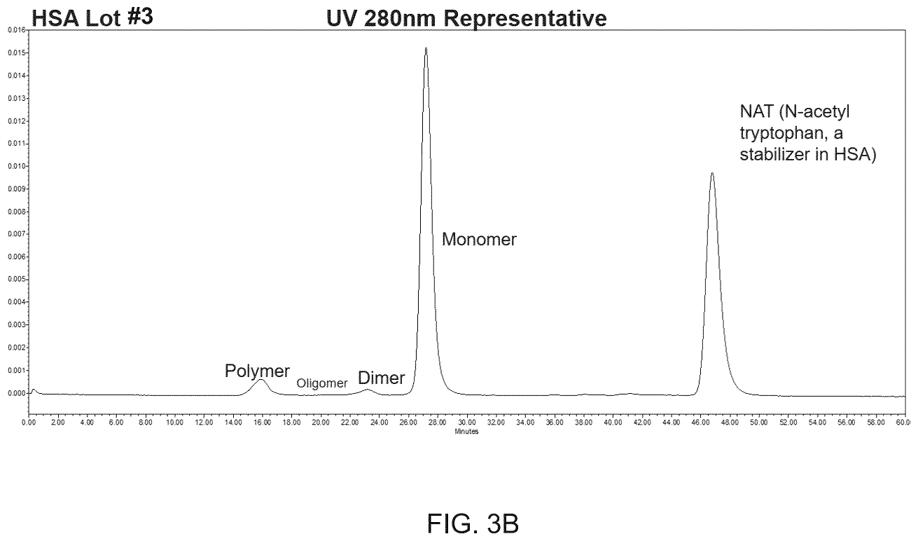

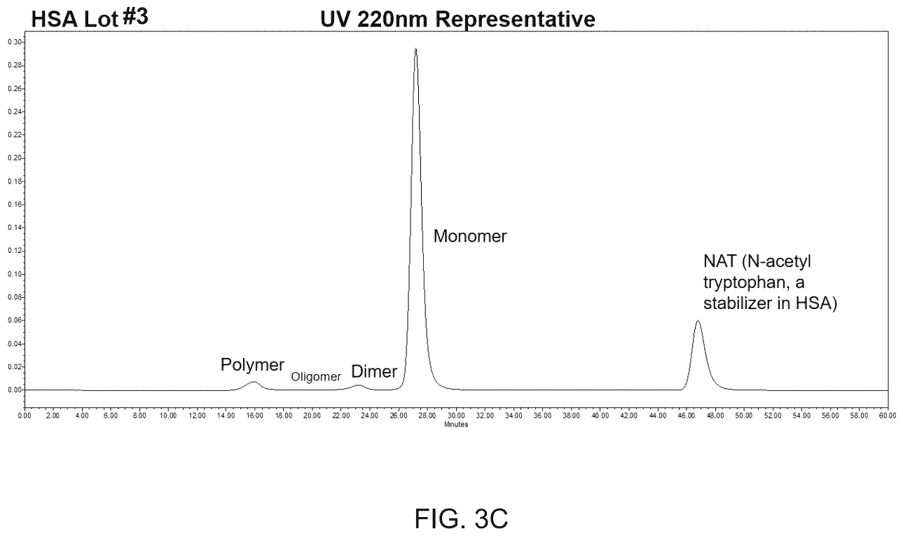

Representative SEC chromatograms for an HSA solution (raw material) are shown in FIGS. 3A-3C. In addition to HSA monomer peak (retention time, RT˜27 min), there were 3 peaks of the higher molecular weight species, identified as dimer (RT˜23.5 min), HSA oligomer (RT˜21 min), and HSA polymer (RT˜16 min). Polymer, oligomer, dimer, and monomer peaks were assigned based on retention times obtained when running Biorad gel filtration standard (Cat 151-1901) on the same column. The gel filtration standard consisted of bovine thyroglobulin (MW 670 kDa), bovine γ-globulin (MW 158 kDa), chicken albumin (MW 44 kDa), horse myoglobulin (MW 17 kDa), and Vitamin B12 (MW 1350 Da). Depending on the detection method, total aggregation in the soluble HSA subjected to SEC chromatography between about 4% to about 10% was observed, with highest values measured by fluorescence detector followed by UV detection at 280 nm wavelength. Oligomers were detected by fluorescence, but not by the UV detector, probably because of the higher sensitivity of the fluorescent detector.

Between three HSA lots used in this study, aggregation levels were similar (Tables 7-9).

TABLE 7
SEC analysis of aggregates in HSA solution (A220 nm).

| | A220 nm | | | |
|---|---|---|---|---|
| | | | | Total |
| HSA Lot# | Polymer (%) | Oligomer (%) | Dimer (%) | aggregates (%) |
| 1 | 2.6 ± 0.5 | 0.0 ± 0.0 | 1.3 ± 0.2 | 3.83 ± 0.06 |
| 2 | 2.4 ± 0.5 | 0.0 ± 0.0 | 1.8 ± 0.2 | 4.2 ± 0.0 |
| 3 | 2.9 ± 0.0 | 0.0 ± 0.0 | 1.3 ± 0.0 | 4.2 ± 0.0 |

TABLE 8
SEC analysis of aggregates in HSA solution (A280 nm).

| | A280 nm | | | |
|---|---|---|---|---|
| | | | | Total |
| HSA Lot# | Polymer (%) | Oligomer (%) | Dimer (%) | aggregates (%) |
| 1 | 4.8 ± 0.9 | 0.0 ± 0.0 | 0.8 ± 0.3 | 5.67 ± 0.15 |
| 2 | 4.5 ± 0.8 | 0.0 ± 0.0 | 1.4 ± 0.7 | 5.9 ± 0.21 |
| 3 | 5.4 ± 0.1 | 0.0 ± 0.0 | 0.0 ± 0.0 | 5.33 ± 0.12 |

TABLE 9
SEC analysis of aggregates in HSA solution (fluorescence).

| | Fluorescence | | | |
|---|---|---|---|---|
| | | | | Total |
| HSA Lot# | Polymer (%) | Oligomer (%) | Dimer (%) | aggregates (%) |
| 1 | 8.5 ± 1.4 | 0.2 ± 0.1 | 1.8 ± 0.1 | 10.4 ± 0.1 |
| 2 | 7.8 ± 1.0 | 0.2 ± 0.1 | 1.9 ± .1 | 9.9 ± 0.07 |
| 3 | 9.7 ± 0.1 | 0.2 ± 0.0 | 1.8 ± 0.0 | 11.63 ± 0.06 |

### 7.1.3.2. HSA Aggregation in the Active DP Lots and Placebo Lots.

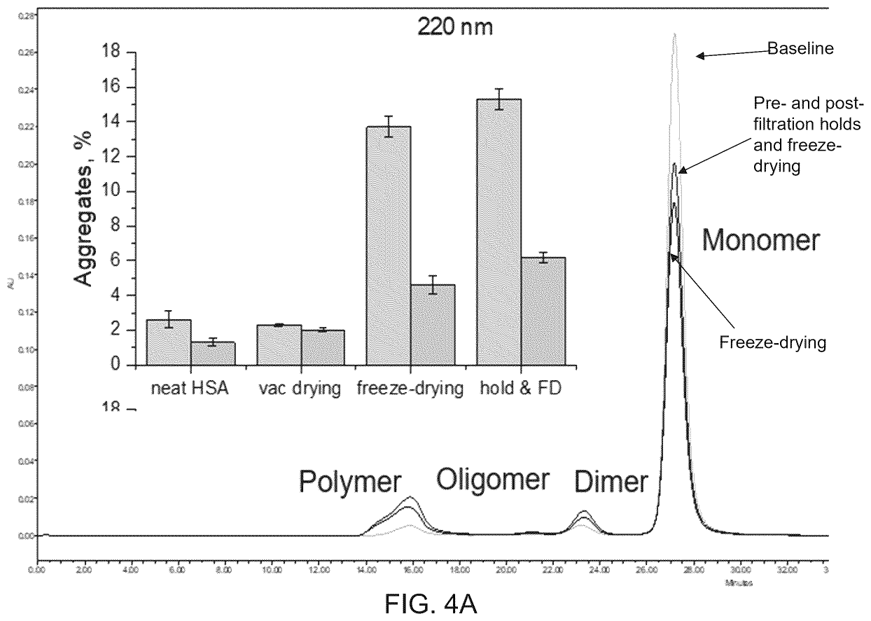

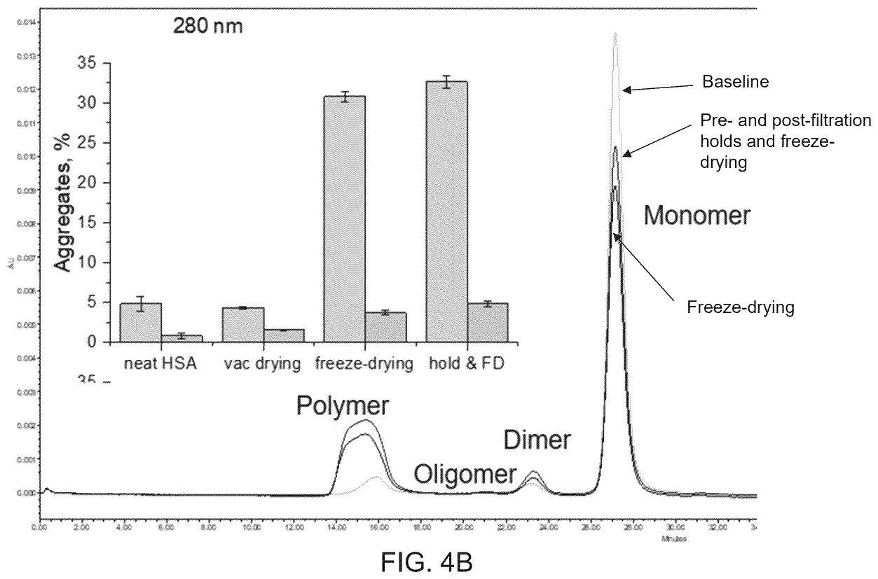

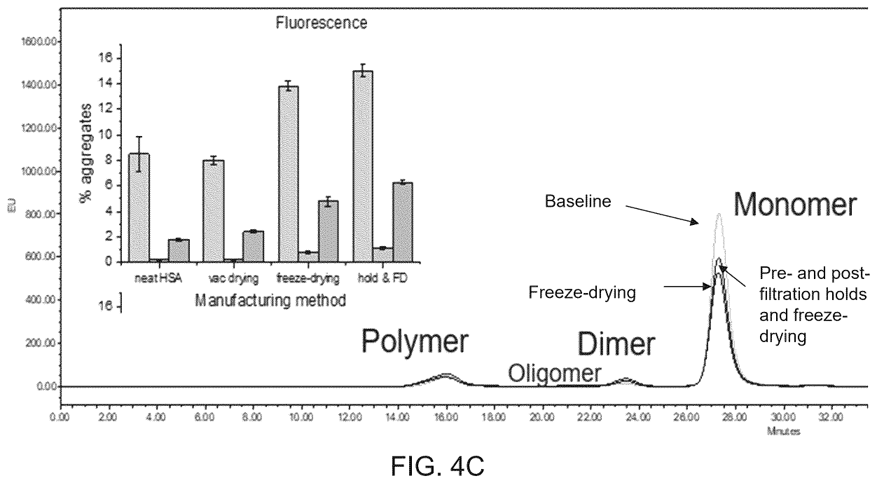

The aggregation of HSA in BoNT/A DP lots was characterized using SEC. Examples of SEC traces for drug product lots produced with three different manufacturing methods are shown in FIGS. 4A-4C. A significant decrease in the peak was observed in both freeze-dried lots produced using process-2 and process-3, as compared with the vacuum-dried lot (process-1). Peaks of HSA dimer and polymer were stronger in freeze-dried samples than in the vacuum dried product. In addition, two poorly-resolved polymer peaks were observed in the freeze-dried samples, especially with the UV detection at both 220 and 280 nm detection wavelengths.

The inset bar graphs in FIGS. 4A-4C show the percent of different types of HSA aggregates in the soluble HSA subjected to SEC chromatography for three DP lots as well as for the “neat” HSA. The values shown in FIGS. 4A-4C are presented in Tables 10-12, below, respectively. Levels of all three types of aggregates were similar between the “neat” HSA and the vacuum-dried DP (manufacture process-1), while there was a significant increase in aggregation for both freeze-dried DP lots. The quantitative extent of aggregation detected depended on the type of detector (see FIGS. 4A-4C and Tables 10-12).

TABLE 10
Percent of different types of HSA aggregation (A220 nm).

| | | polymer % | | dimer % |
|---|---|---|---|---|
| | polymer % | error (SD) | dimer % | error (SD) |
| Neat HSA | 2.6 | 0.5 | 1.3 | 0.2 |
| Vacuum drying | 2.3 | 0.1 | 2 | 0.1 |
| Freeze-drying | 13.7 | 0.6 | 4.6 | 0.5 |
| Freeze-drying with | 15.3 | 0.6 | 6.2 | 0.3 |
| hold | | | | |

TABLE 11
Percent of different types of HSA aggregation (A280 nm).

| | | polymer % | | dimer % |
|---|---|---|---|---|
| | polymer % | error (SD) | dimer % | error (SD) |
| Neat HSA | 4.8 | 0.9 | 0.8 | 0.3 |
| Vacuum drying | 4.3 | 0.1 | 1.5 | 0.1 |
| Freeze-drying | 30.8 | 0.7 | 3.7 | 0.3 |
| Freeze-drying with | 32.7 | 0.8 | 4.8 | 0.3 |
| hold | | | | |

TABLE 12
Percent of different types of HSA aggregation (fluorescence).

| | | | | oligomer | | dimer |
|---|---|---|---|---|---|---|
| | polymer | polymer | oligomer | % error | dimer | % error |
| | % | % error (SD) | % | (SD) | % | (SD) |
| Neat HSA | 8.5 | 1.4 | 0.2 | 0.1 | 1.8 | 0.1 |
| Vacuum drying | 8 | 0.3 | 0.2 | 0.1 | 2.4 | 0.1 |
| Freeze-drying | 13.8 | 0.4 | 0.8 | 0.1 | 4.8 | 0.4 |
| Freeze-drying with | 15 | 0.5 | 1.1 | 0.1 | 6.3 | 0.2 |
| hold | | | | | | |

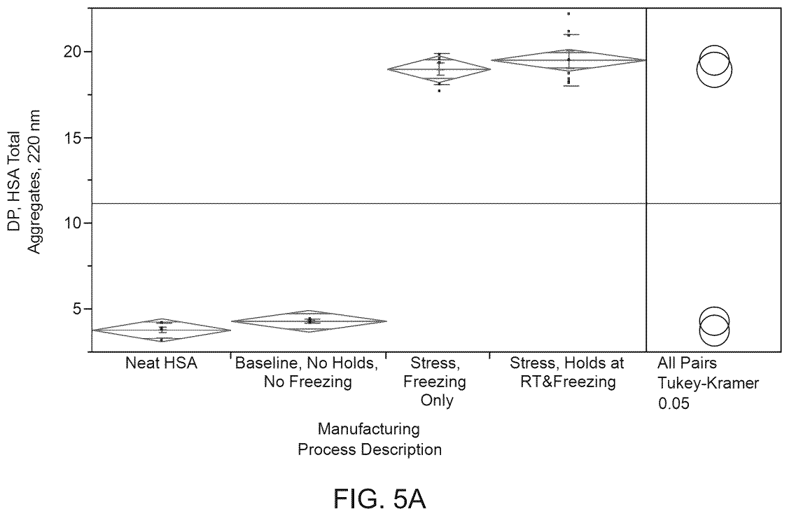

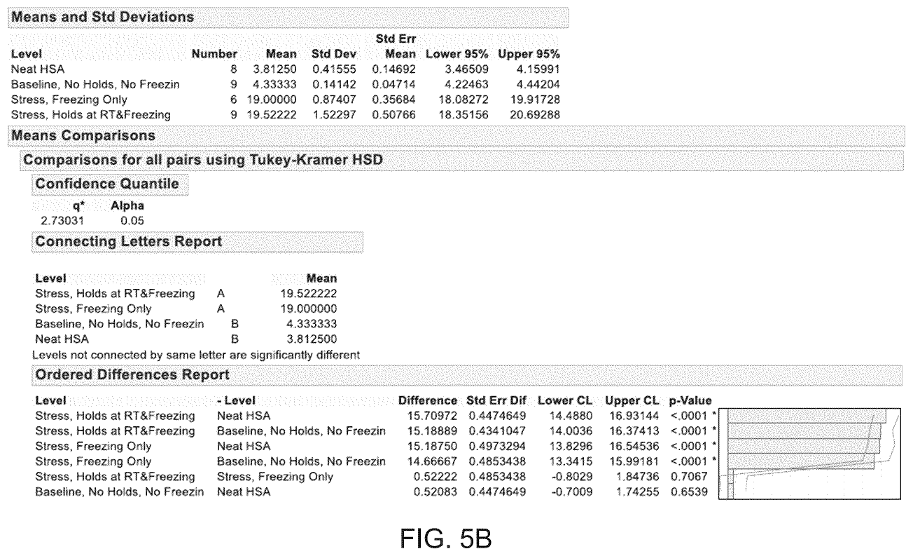

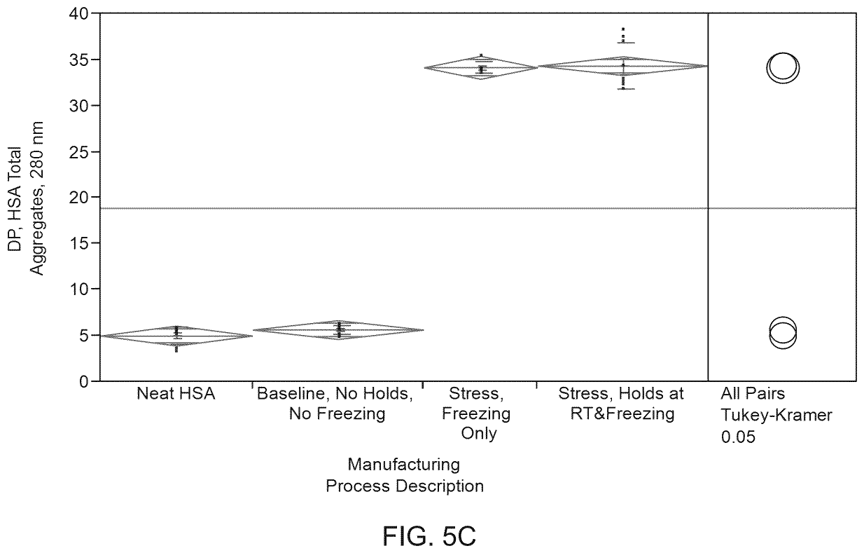

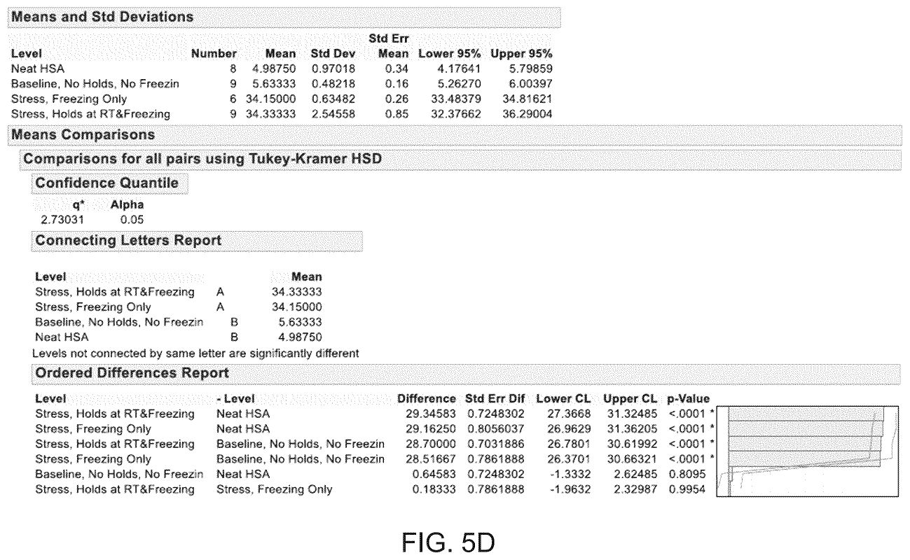

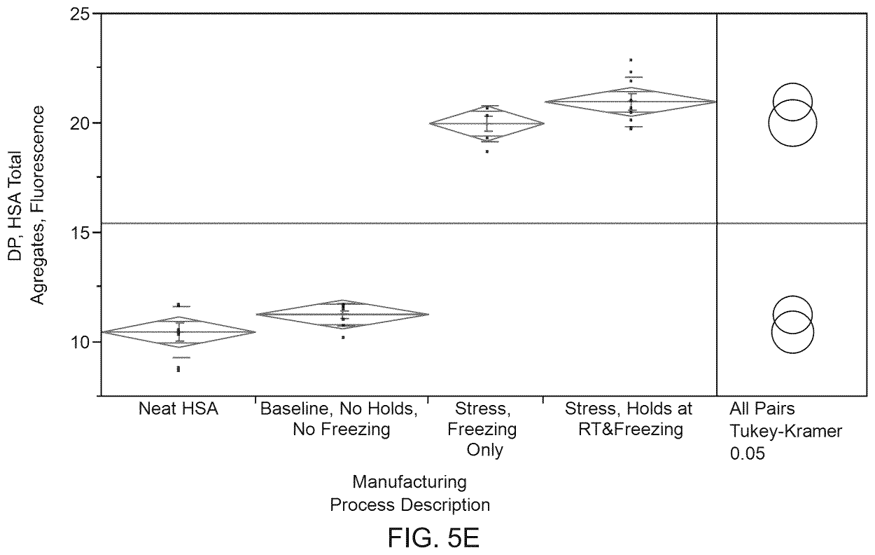

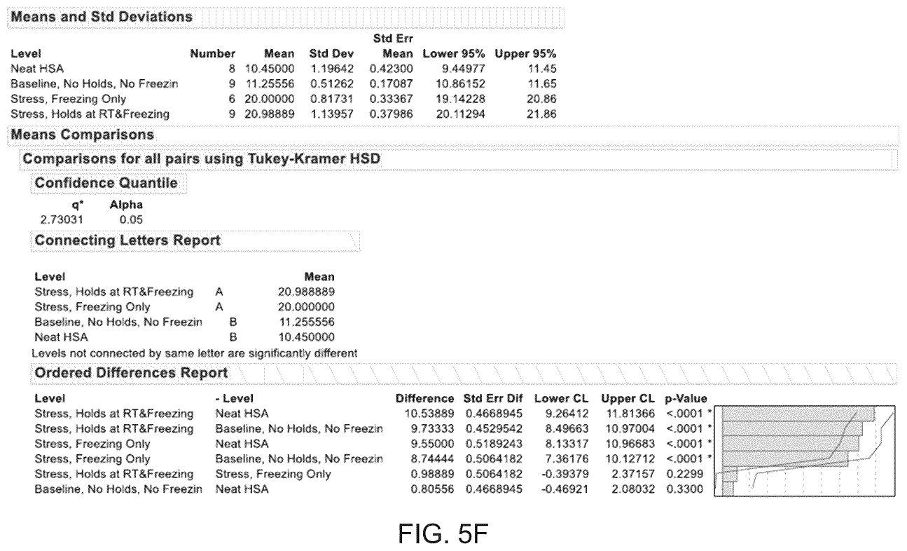

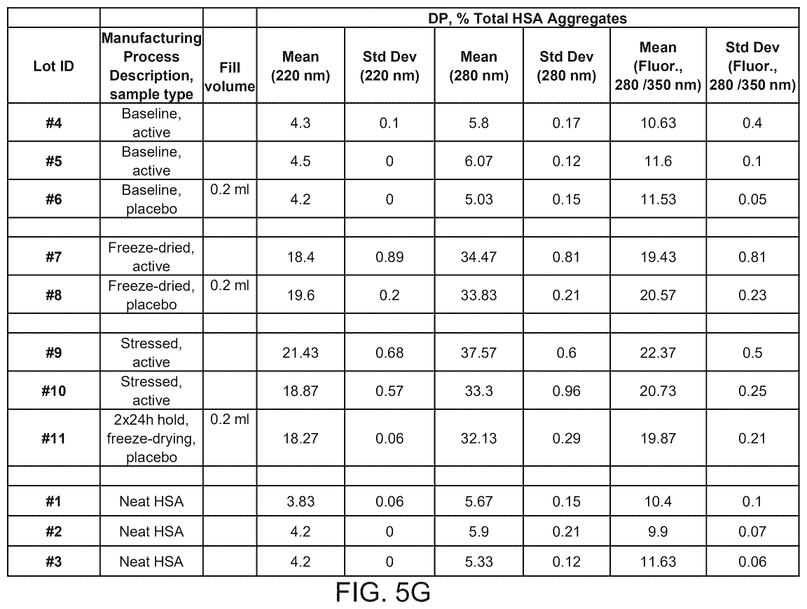

Total HSA aggregation representing the sum of all three types of aggregates in the soluble HSA subjected to SEC chromatography for multiple DP lots is shown in FIGS. 5A-5G. There was no significant increase in aggregation in DP lots produced with manufacturing process-1 (vacuum drying), while the DP lot produced with manufacturing process-2 (freeze-drying) showed a significant increase in HSA aggregation. The longer pre-drying hold time (process-3) did not lead to an additional significant increase in the HSA aggregation.

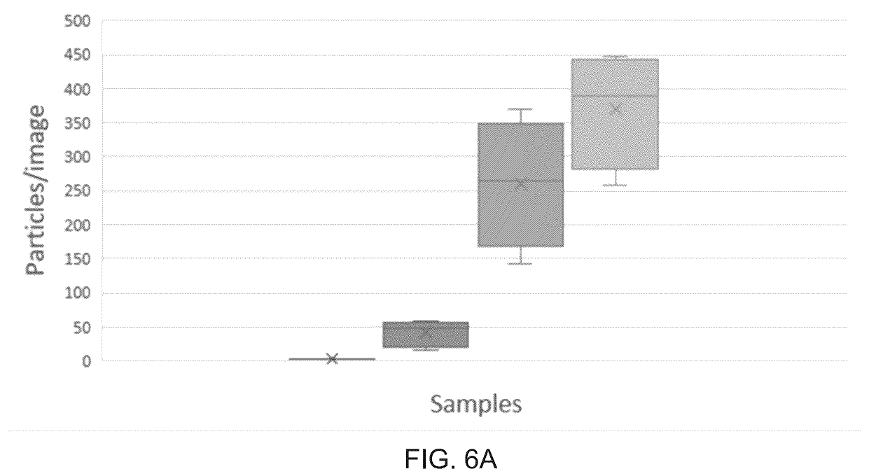

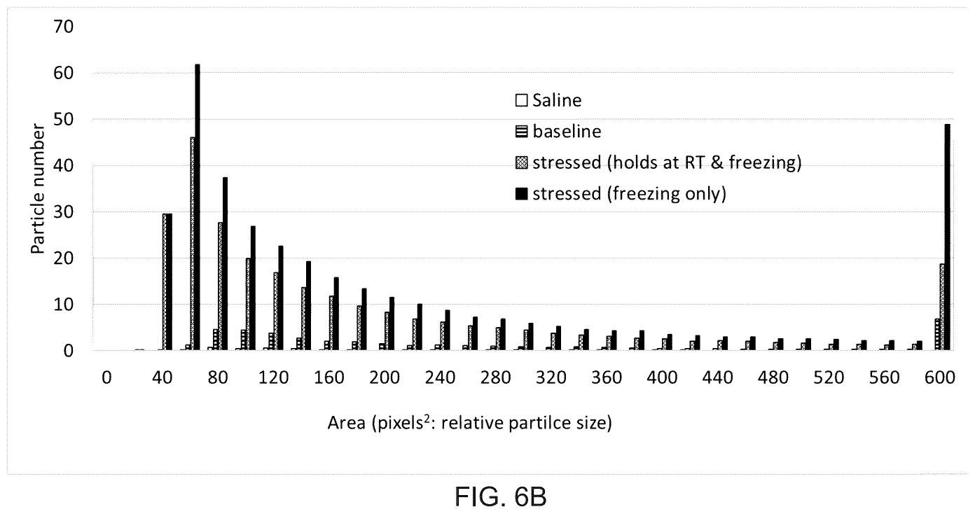

Subvisible particle test was performed with dried placebo lots. The particle counting results are presented in FIGS. 6A and 6B and Table 13. Total average particle count was lower in the vacuum dried lot, while both freeze-dried lots had significantly higher levels of the subvisible particles. Between the two freeze-dried lots, the average particle numbers were similar within the experimental uncertainty. Results on subvisible particles are consistent with SEC data for soluble HSA aggregates (FIGS. 5A-5G), in which aggregation level was higher in the freeze-dried samples as compared with the vacuum-dried lots.

TABLE 13
Subvisible particle test results.

| | | | | Stressed (holds at RT | | Stressed (freezing | |
|---|---|---|---|---|---|---|---|
| Saline | | Baseline (lot #6) | | and freezing) (lot #11) | | only) (lot #8) | |
| Mean | Standard | Mean | Standard | Mean | Standard | Mean | Standard |
| particle # | deviation | particle # | deviation | particle # | deviation | particle # | deviation |
| (n = 1) | (SD) | (n = 5) | (SD) | (n = 4) | (SD) | (n = 4) | (SD) |
| 2.867 | | 40.1802 | 19.71 | 260.067 | 94.77 | 370.459 | 85.93 |

### 7.1.3.3. Physical Characterization of the Drug Product

In order to obtain insights into potential mechanisms leading to the increase in aggregation of HSA during freeze-drying, additional physical tests were performed.

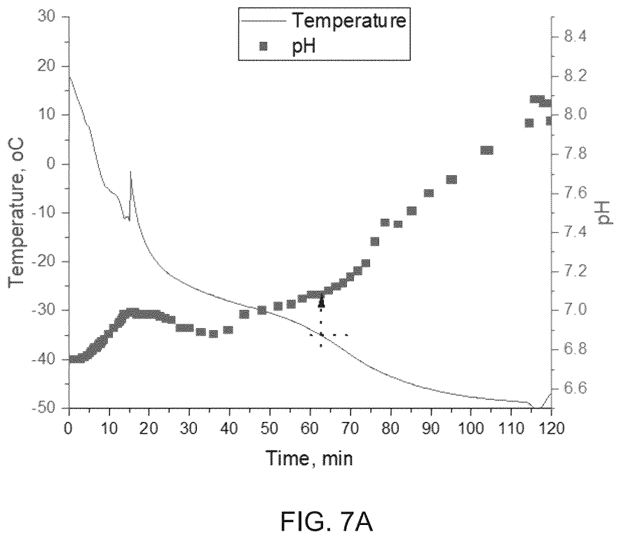

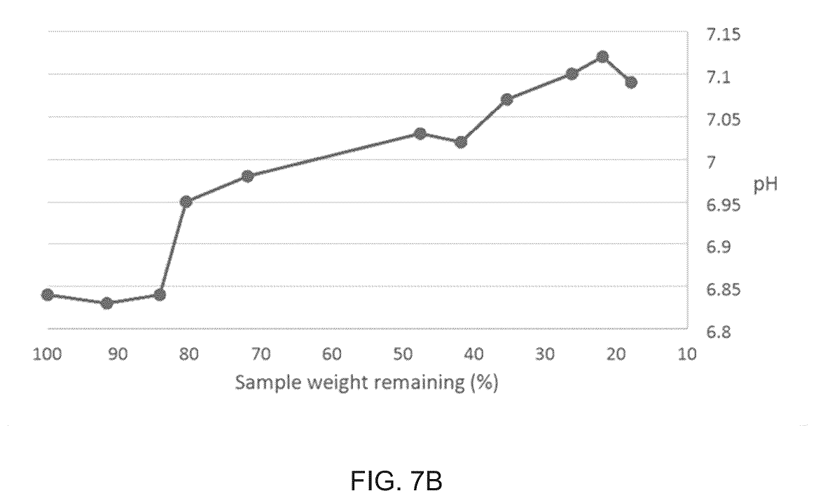

Apparent pH during freezing was measured using low-temperature pH electrodes InLab®Cool (pH 1 to 11, temperature −30° C. to 80° C., Electrolyte PRISCOLYT-B®) (METTLER TOLEDO). The results are presented in FIGS. 7A and 7B. A relatively minor but noticeable increase in pH from initial pH 6.7 to 7 was observed during cooling of the solution from RT to −10° C. Ice nucleation, which was detected at −10° C. as the product temperature spike, did not result in any noticeable pH change. During further cooling to −35° C., pH remained between 6.9 and 7.1, while pH increased to approximately 8.1 during cooling from −35° C. to −50° C. This increase in pH may be related to the secondary crystallization NaCl*2H₂O+water, which was detected at −35° C. in a separate WAXS experiment. While the product temperature did not indicate any evidence of exothermic effect due to the secondary crystallization, the lack of the thermal effect could be due to sensitivity due to low concentration of NaCl (0.9 wt %). Note however that the temperature range of the pH electrode, per the manufacturer specification, extends to −30° C. only; therefore the pH results below −30° C. would need to be confirmed by an orthogonal method. In the evaporation experiment, a minor pH increase, from 6.8 to 7.1, was observed, with the total solute concentration increasing from 1.4 wt % in the initial solution to approximately 8 wt %.

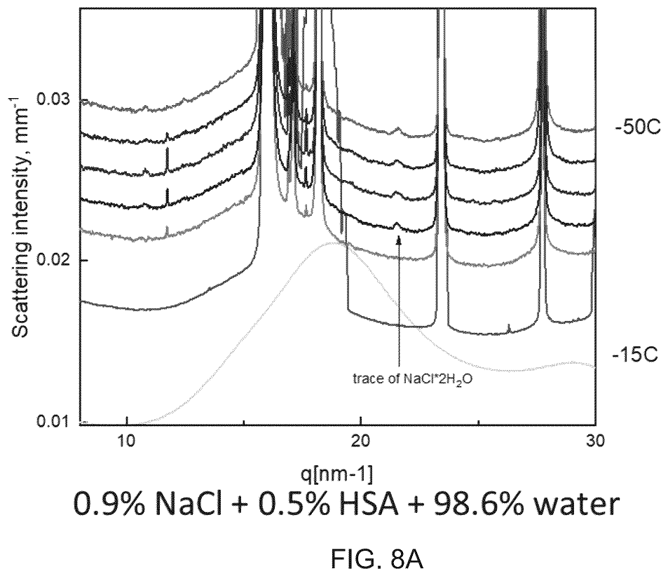

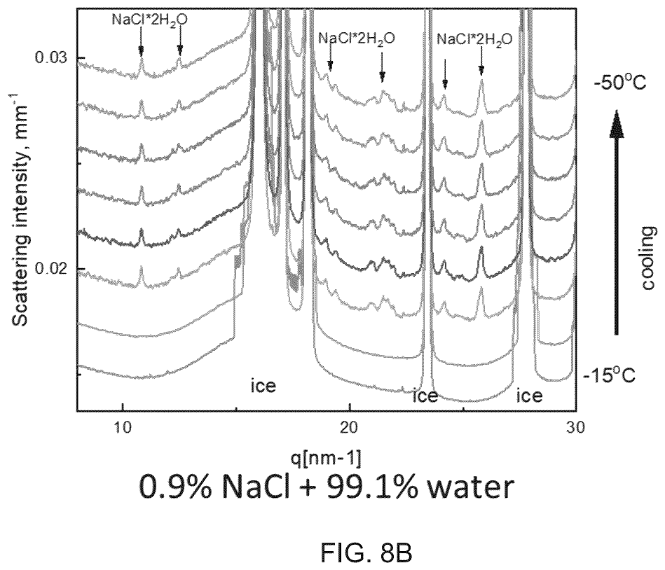

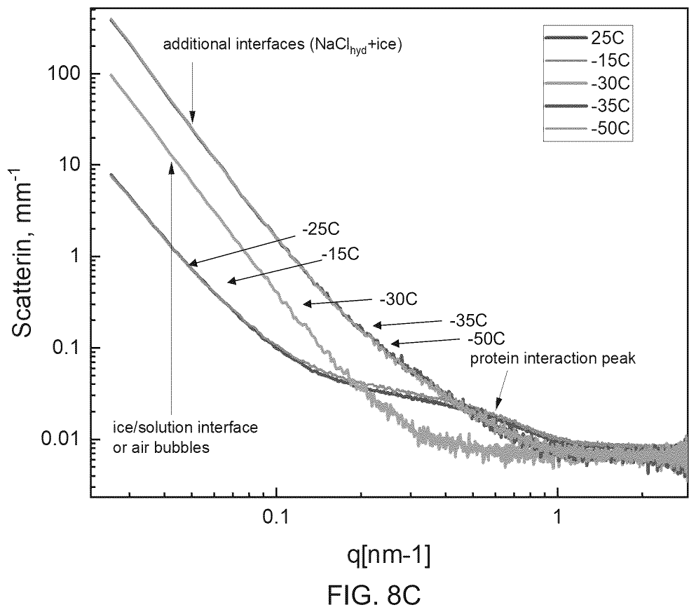

Phase transitions in water/NaCl and water/NaCl/HSA solutions during cooling were studied by small-angle and wide-angle X-ray scattering (SAXS/WAXS). Two aqueous placebo solutions were tested, one containing 0.9% NaCl and 0.5% HSA, and another containing only 0.9% NaCl. The WAXS data showed the formation of hexagonal ice at either −20° C. (HSA+NaCl solution) or −15° C. (NaCl solution) (FIGS. 8A and 8B). Upon further cooling, characteristic peaks of NaCl dihydrate were detected at −35° C. (HSA+NaCl solution) and at −25° C. (NaCl solution). The WAXS results indicate that HSA may hinder crystallization of NaCl dihydrate.

SAXS patterns are shown in FIG. 8C. Protein interaction peak was observed in unfrozen solution, while freezing results in the peak's “disappearance”. In addition to changes of the protein interaction peak, freezing, which was observed by WAXS at −20° C., led to an increase in the low-angle scattering intensity, which was indicative of the formation of additional interfaces, i.e., ice/solution and probably ice/air/solution interfaces. Further cooling to −35° C. led to an additional increase in low-angle scattering, which was indicative of the formation of new interfaces; this increase in the interface coincided with the crystallization of NaCl*2H₂O as observed by WAXS.

Two approaches were used to confirm that the material did not freeze during vacuum drying (manufacturing process-1): monitoring product temperature during vacuum drying, and testing dried products using scanning electron microscopy (SEM).

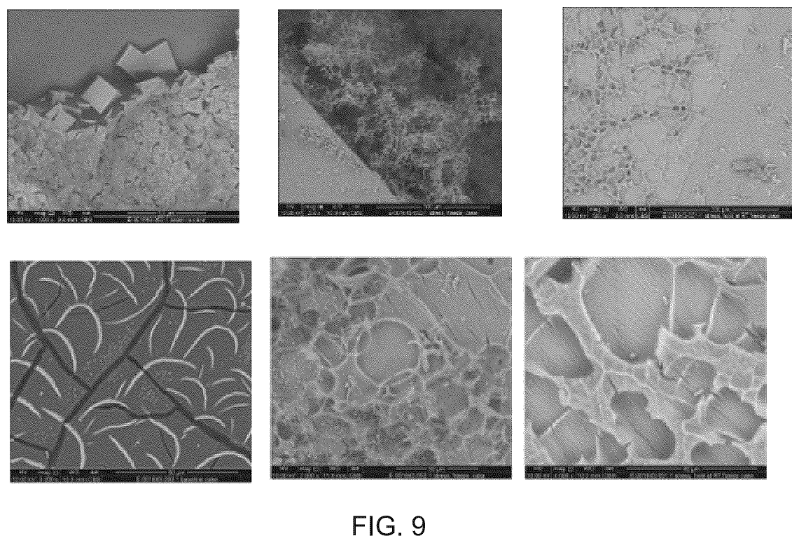

Representative product temperature data (FIG. 2A) show that the temperature remained above 0° C. during the entire vacuum drying cycle; therefore, freezing would not take place in the vials. To further confirm the absence of freezing during vacuum-drying, SEM tests were performed on materials in vials without thermocouples. SEM images for freeze-dried products (FIG. 9) demonstrate the porous morphology of the two freeze-dried lots (right and central images), as expected. Pores in dried materials represent a “signature” of ice crystals which were sublimed during primary drying. The absence of pores in the vacuum-dried material (left images) confirmed that these materials did not go through freezing. Another major difference between vacuum-dried and freeze-dried samples is the appearance of NaCl crystals, which occurred as cubic shapes lining the inner walls of cracks in the HSA in the vacuum dried material. The freeze-dried lots did not have any clear signs of NaCl crystals, possibly because they were much smaller than in the vacuum dried lot. The smaller crystalline size is consistent with XRPD results (below), which show broader peaks for the freeze-dried lot as would be expected for smaller and more disordered crystals.

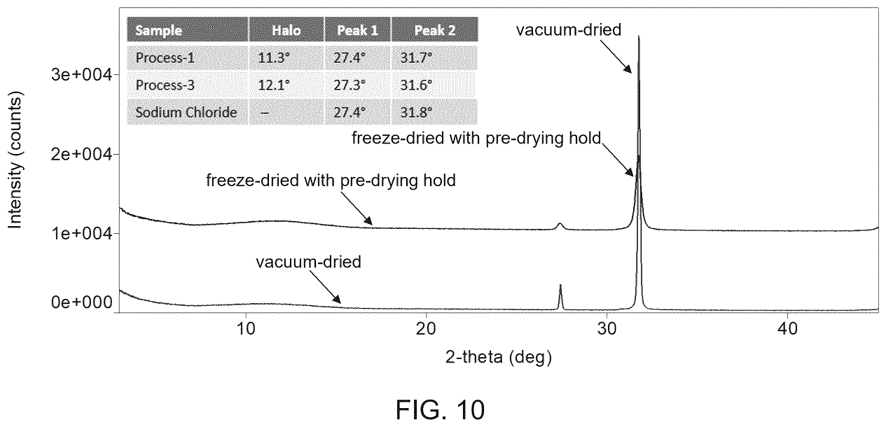

XRPD patterns for two placebo lots, which were produced by different manufacturing methods, are shown in FIG. 10. Two sharp peaks of crystalline NaCl and an amorphous HSA halo were observed in both lots. The vacuum-dried lot (process-1) had stronger and sharper crystalline peaks, while the NaCl crystalline peaks were broader in the freeze-dried sample (process-3), likely reflecting a greater degree of disorder. This result is consistent with the SEM data showing larger NaCl crystals in the vacuum-dried lot. In addition, anhydrous NaCl crystals were formed by dehydration of NaCl dihydrate during freeze-drying, while NaCl crystallized directly from solution during vacuum drying; this difference may also contribute to the higher degree of NaCl crystals disorder in the freeze-dried material.

### 7.1.3.4. Potency Stability Study

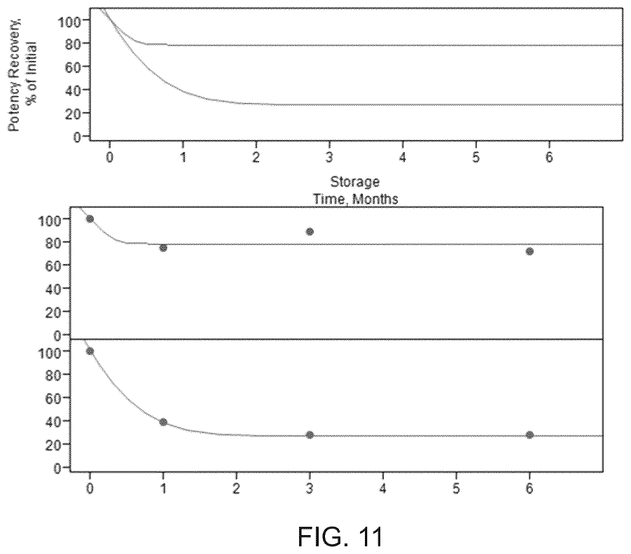

In a separate study, freezing during vacuum drying did seem to result in statistically significant change in BoNT/A DP potency after 6 months of storage at 5° C. as compared to a process without freezing (data not shown). The study of this example further tested potency stability at room temperature and higher in addition to at 5° C. Specifically, two DP lots, lot #9 (process-3, freeze-dried plus hold) and lot #4 (process-1, vacuum-dried), were tested for stability at 5° C., 25° C. and 40° C., respectively. Vials from each lot were pulled at various intervals between 0 and 6 months and analyzed for potency recovery by cell-based potency assay (CBPA). Results are shown in Table 14 below. Data from the test at the 25° C. storage condition are also shown in FIG. 11.

TABLE 14
Results of the potency stability study.

| | | | Storage | | | Potency |
|---|---|---|---|---|---|---|
| DP | Drying | Storage | Time, | Target Potency | Measured Potency | Recovery, % |
| lot # | Process | Condition | Months | Recovery, U/Vial | Recovery, U/Vial | of Initial |
| 9 | Process 3 | 5 C. | 0 | 100 | 73 | 100 |
| 9 | Process 3 | 5 C. | 6 | 100 | 44 | 60.27 |
| 4 | Process 1 | 5 C. | 0 | 100 | 153 | 100 |
| 4 | Process 1 | 5 C. | 6 | 100 | 190 | 124.18 |
| 9 | Process 3 | 25 C., 60% RH | 0 | 100 | 73 | 100 |
| 9 | Process 3 | 25 C., 60% RH | 1 | 100 | 28 | 38.36 |
| 9 | Process 3 | 25 C., 60% RH | 3 | 100 | 20 | 27.4 |
| 9 | Process 3 | 25 C., 60% RH | 6 | 100 | 20 | 27.4 |
| 4 | Process 1 | 25 C., 60% RH | 0 | 100 | 153 | 100 |
| 4 | Process 1 | 25 C., 60% RH | 1 | 100 | 115 | 75.16 |
| 4 | Process 1 | 25 C., 60% RH | 3 | 100 | 136 | 88.89 |
| 4 | Process 1 | 25 C., 60% RH | 6 | 100 | 110 | 71.9 |
| 9 | Process 3 | 40 C., 75% RH | 0 | 100 | 73 | 100 |
| 9 | Process 3 | 40 C., 75% RH | 1 | 100 | 8 | 10.96 |
| 9 | Process 3 | 40 C., 75% RH | 3 | 100 | 6 | 8.22 |
| 9 | Process 3 | 40 C., 75% RH | 6 | 100 | 2 | 2.74 |
| 4 | Process 1 | 40 C., 75% RH | 0 | 100 | 153 | 100 |
| 4 | Process 1 | 40 C., 75% RH | 1 | 100 | 132 | 86.27 |
| 4 | Process 1 | 40 C., 75% RH | 3 | 100 | 117 | 76.47 |
| 4 | Process 1 | 40 C., 75% RH | 6 | 100 | 68 | 44.44 |

RH: relative humidity (designed to simulate storage under accelerated conditions).

As shown, while potency of the DP lot produced by process-3 slightly declined at 5° C. after 6 months, it dramatically declined at 25° C. and 40° C. after even just 3 months. In contrast, potency of the DP lot produced by process-1 declined much slower at 25° C. and 40° C.

### 7.1.4. Discussion

Elevated level of HSA aggregation was observed in freeze-dried lots, while vacuum drying without freezing did not lead to a significant increase in HSA aggregation. HSA aggregation did not increase during vacuum drying when freezing was avoided. The level of soluble aggregates increased from the initial about 4% to about 10% in the “neat” HSA to about 20% to about 35% (specific values depend on the SEC detector, see FIGS. 5A-5G) after freeze-drying and reconstitution. Insoluble aggregates also increased significantly in freeze-dried materials relative to vacuum-dried materials. The results show that, by precisely controlling specific manufacturing conditions, the aggregation of HSA can be significantly reduced, with lower levels of both soluble and insoluble HSA aggregates detected when freezing is avoided during drying. The results also indicate that freezing during drying may lead to faster potency decline during storage.

Protein destabilization and aggregation could potentially relate to protein concentration and ionic strength. However, the lack of HSA aggregation in the vacuum dried product lots indicate that neither an increase in protein concentration nor ionic strength were the main mechanisms of the observed HSA aggregation per se.

Without wishing to be bound by any one theory, the results of this study suggest that the observed HSA aggregation may relate to NaCl*2H₂O. In particular, secondary crystallization (NaCl*2H₂O+water) could create favorable conditions for freeze-induced pressure, as it takes place in a rigid “case” of ice crystals, which was formed during primary freezing event, and thereby possibly contributing to protein destabilization during freezing. Crystallization of NaCl*2H₂O may also contribute to HSA aggregation via a different mechanism. While crystallization of anhydrous NaCl during vacuum-drying did not lead to HSA aggregation, a different NaCl crystal form, dihydrate, was formed during freeze-drying. Anhydrous NaCl formed during vacuum drying may have different surface properties from the dihydrate formed during freezing. While the surface charge on NaCl and NaCl*2H₂O crystals formed during vacuum-drying and freeze-drying, respectively, is not known, a significant difference in the electrostatic interaction of these two types of crystals with HSA may be a contributing factor in freeze-induced HSA aggregation.

Finally, pH changes were observed in this study during freezing, which could also contribute to the HSA aggregation.

### 7.1.5. Conclusions

While the majority of commercial neurotoxin formulations contain HSA, there is a lack of understanding of the impact of drug product manufacturing process on HSA properties. In this study, a significant increase in the aggregation of HSA was observed during freeze-drying, while the aggregation of HSA in vacuum-dried DP without freezing did not increase significantly and remained similar to that in the initial HSA solution. These observations suggest that manufacturing conditions of neurotoxin formulations may have a significant impact on the properties of the finished product. Therefore, additional testing of toxin DP, in particular HSA aggregation, may be beneficial in the development and commercialization of HSA-containing neurotoxin products. The results further indicate that freezing was the major root cause of HSA aggregation.

On mechanism of aggregation of HSA during freeze-drying, the study indicates that freeze-induced pressure, interaction with crystals of NaCl*2H₂O, and freeze-induced pH changes could be contributing factors.

### 7.2. Example 2—Impact of Freezing on Vacuum Dried Botulinum Toxin Drug Products

In this study, BoNT/A DP lots produced by a vacuum drying process that did not involve freezing of the drug product solutions were compared with BoNT/A DP lots produced by a vacuum drying process that induced freezing of the drug product solutions. The two types of BoNT/A DP lots are referred to in this example as evaporative vacuum dried DP lots and vacuum induced lyophilized DP lots, respectively. Both the pilot scale freeze dryer Lyostar-3 and the industrial scale freeze dryer Lyomax 40 were tested. For both freeze dryers, the evaporative vacuum dried DP lots were repeatedly observed to have a more visually homogenous morphology leading to consistent visual product quality for the patient, while vacuum induced lyophilized DP lots were prone to flaking. A flaked product has the potential to migrate near the product stopper during reconstitution of the drug product vial, and thus may result in the patient getting less than the full prescribed dosage. Thus, evaporative vacuum dried DP lots are more favorable than vacuum induced lyophilized DP lots.

Additional results from this study showed that using a slower chamber depressurization rate and a higher shelf temperature (using a shelf temperature of 20° C. instead of 5° C.) can help prevent freezing of the drug product (although the exact chamber depressurization rate to be employed would be unique to the drying equipment and would depend on chamber size, condenser capacity, chamber load, and vacuum pump performance). Results from this study showed that both fast chamber depressurization rate and low shelf temperature were significant factors contributing to lower product temperature.

### 8. INCORPORATION BY REFERENCE

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A solid composition comprising a 900 kDa Clostridium botulinum neurotoxin serotype A (BoNT/A) complex, human serum albumin (HSA), and sodium chloride (NaCl), wherein the HSA comprises HSA monomer and HSA aggregates, wherein at least a portion of the HSA is in the form of soluble HSA when the solid composition is reconstituted in an aqueous medium, and wherein less than 34% of soluble HSA contained in the solid composition are HSA aggregates.

2. The solid composition of claim 1, wherein the HSA aggregates comprise HSA polymer, HSA oligomer, and HSA dimer.

3. The solid composition of claim 1, wherein less than 30% of the soluble HSA contained in the solid composition is HSA polymer.

4. The solid composition of claim 1, wherein less than 0.8% of soluble HSA contained in the solid composition is HSA oligomer.

5. The solid composition of claim 1, wherein less than 4.8% of soluble HSA contained in the solid composition is HSA dimer.

6. The solid composition of claim 1, wherein the percentage of the HSA aggregates in the soluble HSA contained in the solid composition is determined using size exclusion chromatography (SEC) after at least a portion of the solid composition is reconstituted with water.

7. The solid composition of claim 1, wherein the solid composition is a powdered pharmaceutical composition.

8. The solid composition of claim 1, wherein the percentage of the HSA aggregates in the soluble HSA contained in the solid composition is determined using size exclusion chromatography (SEC) by a 220 nm wavelength UV detector after at least a portion of the solid composition is reconstituted with water.

9. The solid composition of claim 1, wherein the solid composition is animal product free.

10. The solid composition of claim 1, wherein the HSA is recombinant HSA.

11. The solid composition of claim 1, wherein the solid composition comprises about 0.5 mg of HSA per 100 units of BoNT/A.

12. The solid composition of claim 1, wherein the solid composition comprises about 0.9 mg of NaCl per 100 units of BoNT/A.

13. The solid composition of claim 1, wherein the BoNT/A is onabotulinumtoxinA.

14. The solid composition of claim 1, where the BoNT/A is produced by a column chromatography purification process.

15. The solid composition of claim 1, where the BoNT/A is produced by a column chromatography purification process, wherein the column chromatography purification process comprises hydrophobic interaction chromatography.

16. The solid composition of claim 1, wherein the column chromatography purification process further comprises anion exchange chromatography.

17. The solid composition of claim 1, wherein the column chromatography purification process further comprises cation exchange chromatography.

18. The solid composition of claim 1, wherein the solid composition does not comprise a protease inhibitor.

19. The solid composition of claim 1, wherein the solid composition does not comprise benzamidine hydrocholoride.

20. The solid composition of claim 1, wherein the solid composition has a potency of about 1.5×10⁷ units/mg to about 6.0×10⁷ units/mg.

21. A method of producing a solid composition comprising a 900 kDa BoNT/A complex, HSA, and NaCl, said method comprising a step of drying a solution comprising BONT/A, HSA, and NaCl to produce said solid composition, wherein the solution does not undergo freezing during the drying step.

22. The method of claim 21, wherein the step of drying is at least partially manually controlled.

23. The method of claim 21, wherein the step of drying is vacuum drying.

24. The method of claim 23, wherein the vacuum drying step comprises a stepwise evacuating step.

25. (canceled)

26. A solid composition produced by the method of claim 21.

27. A method of treating a patient in need thereof, comprising administering the solid composition of claim 1 to the patient.

28. A method substantially as described herein.

29. A composition substantially as described herein.
